(19) United States
(12) Patent Application Publication
(10) Pub. No.: US 2024/0140943 A1
(43) Pub. Date: May 2, 2024

(54) COMPETITIVE AND NONCOMPETITIVE INHIBITORS OF THE MUSCARINIC ACETYLCHOLINE RECEPTOR M5

(71) Applicant: Vanderbilt University, Nashvilee, TN (US)

(72) Inventors: Craig W. Lindsley, Brentwood, TN (US); P. Jeffrey Conn, Nashville, TN (US); Changho Han, Nashville, TN (US); Jinming Li, I, Nashville, TN (US)

(21) Appl. No.: 18/263,052

(22) PCT Filed: Jan. 26, 2022
(86) PCT No.: PCT/US2022/013804

§ 371 Date: Jul. 26, 2023

Related U.S. Application Data
(60) Provisional application No. 63/141,722, filed on Jan. 26, 2021.

(51) Int. Cl. C07D 471/04; A61P 25/00

(52) U.S. Cl. CPC C07D 471/04; A61P 25/00

(57) ABSTRACT

Arylsulfonamides of 4-heteroaryl-cyclohexyls, and their derivatives, are competitive and non-competitive inhibitors of the muscarinic acetylcholine receptor M5 (mAChR M5) and have utility in the treatment of psychiatric disorders such as substance-related misuse, substance-related disorder relapse, anxiety, depression, and psychosis.

### RELATED APPLICATIONS

This application claims priority to U.S. Provisional Application No. 63/141,722, filed Jan. 26, 2021, which is hereby incorporated by reference in its entirety.

### TECHNICAL FIELD

The present disclosure relates to compounds, compositions, and methods for treating disorders associated with muscarinic acetylcholine receptor subtype 5 dysfunction or disorders that benefit from inhibition of the muscarinic acetylcholine receptor subtype 5.

### BACKGROUND

Substance-related disorders, e.g., opiate use disorder (OUD), alcohol use disorder (AUD), cocaine use disorder (CUD) and nicotine use disorder (NUD), are debilitating neuropsychiatric conditions that involve periods of compulsive drug use, followed by dependence and then repeated instances of relapse after periods of abstinence. Currently, OUD is a global epidemic. Prescription opioid analgesics are effective pain medications; however, the use of opioid analgesics is also associated with high risks of misuse, dependence, and overdose due to their strong rewarding effects. In addition, the vast majority of all estimated drug-related overdose deaths involve opioids, with nearly half of those attributed to prescription pain medications. There is no FDA-approved treatment for OUD.

Recent attention has focused on the M₅ muscarinic acetylcholine receptor (M₅ mAChR) in motivated behaviors, including drug self-administration, and thus inhibition of this receptor may represent an alternative strategy for the reduction or blockade of the reinforcing effects of multiple substances of abuse.

Of the five mAChR subtypes (M₁-M₅) activated by acetylcholine (ACh), the M₅ mAChR has very limited CNS expression, and is the only subtype expressed on dopamine neurons in the ventral midbrain, including the ventral tegmental area (VTA) and the substantia nigra pars compacta (SNc). VTA dopaminergic neurons project to the nucleus accumbens, also known as the canonical mesolimbic reward pathway. All substances of abuse, including opioids and stimulants, increase dopamine release in the nucleus accumbens and drug seeking behaviors. Due to its localization, the M₅ receptor provides important control of midbrain dopaminergic neuronal activity under physiological conditions and after exposure to substances of abuse. Consistent with this supposition, increases in extracellular DA efflux in the nucleus accumbens induced by the μ-opioid agonist morphine were absent in M₅ knockout [KO] mice. Moreover, M₅ KO mice showed significantly reduced reinforcing effects of cocaine as well as opioid place preference. Additionally, severity of naloxone-induced morphine withdrawal symptoms were also reduced in the M₅ KO mice. In contrast, the acute analgesic effects of morphine and the development of tolerance to these effects remained unaltered in the M₅ KO mice relative to the wild-type control mice.

Thus, compounds possessing a more selective profile for individual mAChRs, such as M_{5,} may offer an advantage in substance use disorders, as well as other neuropsychiatric disorders. For example, some studies indicate that the M₅ mAChR subtype may play a therapeutic role in depression and anxiety; however, a lack of highly selective M₅ antagonists has hindered the field.

### SUMMARY

In one aspect, the invention provides compounds of formula (I), or a pharmaceutically acceptable salt thereof,

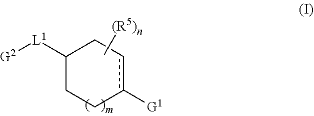

wherein:
“ [symbol] ” is a single or double bond;
m is 0 or 1;
L¹ is SO₂ or SO;
G¹ is a 9- to 12-membered heteroaryl, G¹ being attached at an aromatic ring carbon atom, wherein G¹ is optionally substituted with 1-5 substituents independently selected from the group consisting of oxo, halogen, C₁₋₆alkyl, C₁₋₆haloalkyl, C₂₋₆alkenyl, —OR^{1a}, —NR^{1a}R^{1b}, —SR^{1a}, —NR^{1a}C(O)R^{1c}, cyano, —C(O)OR^{1a}, —C(O)NR^{1a}R^{1b}, —C(O)NR^{1c}, —SO₂R^{1d}, —SO₂NR^{1a}R^{1b}, G^{1a}, —C₁₋₃alkylene-G^{1a}, and —C₁₋₃alkylene-Y¹;
G² is a 6- to 12-membered aryl or 5- to 12 membered heteroaryl, each optionally substituted with 1-5 substituents independently selected from the group consisting of halogen, C₁₋₆alkyl, C₁₋₆haloalkyl, oxo, —OR^{2a}, —NR^{2a}R^{2b}, —SR^{2a}, —NR^{2a}C(O)R^{2c}, cyano, —C(O)OR^{2a}, —C(O)NR^{2a}R^{2b}, —C(O)R^{2c}, —SO₂R^{2d}, —SO₂NR^{2a}R^{2b}, G^{2a}, —C₁₋₃alkylene-G^{2a}, and —C₁₋₃alkylene-Y²;
R^{1a}, R^{1b}, and R^{1c}, at each occurrence, are each independently hydrogen, C₁₋₆alkyl, C₁₋₆haloalkyl, G^{1a}, or —C₁₋₃alkylene-G^{1a};
R^{1d}, at each occurrence, is independently C₁₋₆alkyl, C₁₋₆haloalkyl, G^{1a}, or —C₁₋₃alkylene-G^{1a};
R^{2a}, R^{2b}, and R^{2c}, at each occurrence, are each independently hydrogen, C₁₋₆alkyl, C₁₋₆haloalkyl, G^{2a}, or —C₁₋₃alkylene-G²;
R^{2d}, at each occurrence, is independently C₁₋₆alkyl, C₁₋₆haloalkyl, G^{2a}, or —C₁₋₃alkylene-G^{2a};
G^{1a} and G^{2a}, at each occurrence, are independently a C₃₋₈cycloalkyl, a 4- to 12-membered heterocyclyl, a 6- to 12-membered aryl, or a 5- to 12-membered heteroaryl, wherein G^{1a} and G^{2a} are independently optionally substituted with 1-5 substituents independently selected from the group consisting of halogen, oxo, C₁₋₄alkyl, —OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Y¹ and Y², at each occurrence, are independently —OC₁₋₄alkyl, —OC₁₋₄haloalkyl, OH, NH₂, —NHC₁₋₄alkyl), —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
R⁵, at each occurrence, is independently halogen, cyano, oxo, C₁₋₄alkyl, C₁₋₄haloalkyl, —OR^{5a}, or C₃₋₄cycloalkyl, wherein optionally two R⁵ substituted on non-adjacent ring atoms, taken together with atoms to which they attach, form a C₁₋₃alkylene bridge;
R^{5a}, at each occurrence, is independently hydrogen, C₁₋₄alkyl, C₁₋₄haloalkyl, C₃₋₄cycloalkyl, or —C₁₋₃alkylene-C₃₋₄cycloalkyl; and
n is 0, 1, 2, 3, 4, or 5;
provided the compound is not 2,8,9-trimethyl-5-[4-(phenylsulfonyl)cyclohexyl]thieno[3,2-e][1,2,4]triazolo[1,5-c]pyrimidine.

In another aspect, the invention provides a pharmaceutical composition comprising a compound of formula (1), or a pharmaceutically acceptable salt thereof, and a pharmaceutically acceptable carrier.

In another aspect, the invention provides a method of treating a disorder in a subject, wherein the subject would benefit from inhibition of mAChR M₅, comprising administering to the subject a therapeutically effective amount of a compound of formula (I), or a pharmaceutically acceptable salt or composition thereof.

In another aspect, the invention provides a method for inhibiting mAChR M₅ in a subject, comprising administering to the subject a therapeutically effective amount of a compound of formula (I), or a pharmaceutically acceptable salt or composition thereof.

In another aspect, the invention provides a method for the treatment of a psychiatric disorder comprising administering to a subject in need thereof a therapeutically effective amount of a compound of formula (I), or a pharmaceutically acceptable salt or composition thereof.

In another aspect, the invention provides a compound of formula (I), or a pharmaceutically acceptable salt or composition thereof, for use in the treatment of a psychiatric disorder.

In another aspect, the invention provides a compound of formula (I), or a pharmaceutically acceptable salt or composition thereof, for use in inhibiting mAChR M₅ in a subject.

In another aspect, the invention provides the use of a compound of formula (I), or a pharmaceutically acceptable salt or composition thereof, in the manufacture of a medicament for the treatment of a psychiatric disorder.

In another aspect, the invention provides the use of a compound of formula (I), or a pharmaceutically acceptable salt or composition thereof, in the manufacture of a medicament for inhibiting mAChR M₅ in a subject.

In another aspect, the invention provides a kit comprising a compound of formula (I), or a pharmaceutically acceptable salt or composition thereof, and instructions for use.

### DETAILED DESCRIPTION

Disclosed herein are compounds that are antagonists of the muscarinic acetylcholine receptor M₅ (mAChR M₅), methods of making the compounds, pharmaceutical compositions comprising the compounds, and methods of treating disorders using the compounds and pharmaceutical compositions.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of and consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Tranformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to a group —O-alkyl. Representative examples of alkoxy include, but are not limited to, methoxy, ethoxy, propoxy, 2-propoxy, butoxy and tert-butoxy.

The term “alkyl” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyL n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkoxyalkyl,” as used herein, refers to an alkoxy group, as defined herein, appended to the parent molecular moiety through an alkyl group, as defined herein.

The term “alkoxyfluoroalkyl,” as used herein, refers to an alkoxy group, as defined herein, appended to the parent molecular moiety through a fluoroalkyl group, as defined herein.

The term “alkylene,” as used herein, refers to a divalent group derived from a straight or branched chain hydrocarbon, for example, of 1 to 3 carbon atoms. Representative examples of alkylene include, but are not limited to, —CH₂—, —CD₂-, —CH₂CH₂—, —C(CH₃)(D)-, —CH₂CH₂CH₂—, —CH₂CH₂CH₂—CH₂—, and —CH₂CH₂CH₂—CH₂CH—.

The term “alkylamino,” as used herein, means at least one alkyl group, as defined herein, is appended to the parent molecular moiety through an amino group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl,” as used herein, means at least one amino group, as defined herein, is appended to the parent molecular moiety through an alkylene group, as defined herein.

The term “amino,” as used herein, means —NRₓR, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ—, wherein Rₓ may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cyanoalkyl,” as used herein, means at least one —CN group, is appended to the parent molecular moiety through an alkylene group, as defined herein.

The term “cyanofluoroalkyl,” as used herein, means at least one —CN group, is appended to the parent molecular moiety through a fluoroalkyl group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1.]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl”' is used herein to refer to a cycloalkene when present as a substituent. A cycloalkenyl may be a monocyclic cycloalkenyl (e.g., cyclopentenyl), a fused bicyclic cycloalkenyl octahydronaphthalenyl), or a bridged cycloalkenyl in which two non-adjacent atoms of a ring are linked by an alkylene bridge of 2, 3, or 4 carbon atoms (e.g., bicyclo[2.2.1]heptenyl). Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The term “fluoroalkyl,” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, ditluoromethyl, pentafluoroethyl, and trifluoropropyl such as 3,3,3-trifluoropropyl.

The term “fluoroalkoxy,” as used herein, means at least one fluoroalkyl group, as defined herein, is appended to the parent molecular moiety through an oxygen atom. Representative examples of fluoroalkoxy include, but are not limited to, difluoromethoxy, trifluoromethoxy and 2,2,2-trifluoroethoxy.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered aromatic monocyclic rings have three double bonds. The bicyclic heteroaryl is an 8- to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienvl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triaziyvl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatoin independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyis include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, and 3-oxaspiro[5.5]undecanyl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta.[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyis are connected to the parent molecular moiety at a non-aromatic ring atom.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

The term “hydroxyfluoroalkyl,” as used herein, means at least one —OH group, is appended to the parent molecular moiety through a fluoroalkyl group, as defined herein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows, Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroaiylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylainino, sulfinylamino, sulfonyl, alkylsulfonyl, arylsulfonyl, aminosulfonyl, sulfinyl, —COOH, ketone, amide, carbamate, and acyl.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

The term “mAChR M₅ receptor negative allosteric modulator” as used herein refers to an agent that binds to an allosteric site on the M₅ receptor and decreases the affinity and/or efficacy of acetylcholine, e.g., a noncompetitive inhibitor.

The term “allosteric site” as used herein refers to a ligand binding site that is topographically distinct from the orthosteric binding site.

The term “orthosteric site” as used herein refers to the primary binding site on a receptor that is recognized by the endogenous ligand or agonist for that receptor. For example, the orthosteric site in the mAChR M₅ receptor is the site that acetylcholine binds to. Compounds of the instant invention display both competitive and noncompetitive modes of M₅ inhibition

For the recitation of numeric ranges herein, each intervening number there between with the same degree of precision is explicitly contemplated. For example, for the range of 6-9, the numbers 7 and 8 are contemplated in addition to 6 and 9, and for the range 6,0-7.0, the number 6.0, 6.1, 6.2, 6.3, 6.4, 6,5, 6.6, 6.7, 6.8, 6.9, and 7.0 are explicitly contemplated.

### 2. COMPOUNDS

In one aspect, the invention provides compounds of formula (1), wherein R⁵, m, and n are as defined herein.

Unsubstituted or substituted rings (i.e., optionally substituted) such as aryl, heteroaryl, etc. are composed of both a ring system and the ring system's optional substituents. Accordingly, the ring system may be defined independently of its substituents, such that redefining only the ring system leaves any previous optional substituents present. For example, a 5- to 12-membered heteroaryl with optional substituents may be further defined by specifying the ring system of the 5- to 12-membered heteroaryl is a 5- to 6-membered heteroaryl (i.e., 5- to 6-membered heteroaryl ring system), in which case the optional substituents of the 5- to 12-membered heteroaryl are still present on the 5- to 6-membered heteroaryl, unless otherwise expressly indicated.

In the following, embodiments of the invention are disclosed. The first embodiment is denoted E1, the second embodiment is denoted E2 and so forth.

E1. A compound of formula (1), or a pharmaceutically acceptable salt thereof,

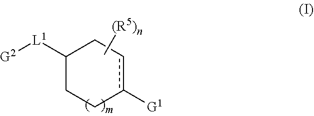

wherein:
“ [symbol] ” is a single or double bond;
m is 0 or 1;
L¹ is SO₂ or SO;
G¹ is a 9- to 12-membered heteroaryl, G¹ being attached at an aromatic ring carbon atom, wherein G¹ is optionally substituted with 1-5 substituents independently selected from the group consisting of oxo, halogen, C₁₋₆alkyl, C₁₋₆haloalkyl, C₂₋₆alkenyl, —OR^{1a}, —NR^{1a}R^{1b}, —SR^{1a}, —NR^{1a}C(O)R^{1c}, cyano, —C(O)OR^{1a}, —C(O)NR^{1a}R^{1b}, —C(O)NR^{1c}, —SO₂R^{1d}, —SO₂NR^{1a}R^{1b}, G^{1a}, —C₁₋₃alkylene-G^{1a}, and —C₁₋₃alkylene-Y¹;
G² is a 6- to 12-membered aryl or 5- to 12 membered heteroaryl, each optionally substituted with 1-5 substituents independently selected from the group consisting of halogen, C₁₋₆alkyl, C₁₋₆haloalkyl, oxo, —OR^{2a}, —NR^{2a}R^{2b}, —SR^{2a}, —NR^{2a}C(O)R^{2c}, cyano, —C(O)OR^{2a}, —C(O)NR^{2a}R^{2b}, —C(O)R^{2c}, —SO₂R^{2d}, —SO₂NR^{2a}R^{2b}, G^{2a}, —C₁₋₃alkylene-G^{2a}, and —C₁₋₃alkylene-Y²;
R^{1a}, R^{1b}, and R^{1c}, at each occurrence, are each independently hydrogen, C₁₋₆alkyl, C₁₋₆haloalkyl, G^{1a}, or —C₁₋₃alkylene-G^{1a};
R^{1d}, at each occurrence, is independently C₁₋₆alkyl, C₁₋₆haloalkyl, G^{1a}, or —C₁₋₃alkylene-G^{1a};
R^{2a}, R^{2b}, and R^{2c}, at each occurrence, are each independently hydrogen, C₁₋₆alkyl, C₁₋₆haloalkyl, G^{2a}, or —C₁₋₃alkylene-G²;
R^{2d}, at each occurrence, is independently C₁₋₆alkyl, C₁₋₆haloalkyl, G^{2a}, or —C₁₋₃alkylene-G^{2a};
G^{1a} and G^{2a}, at each occurrence, are independently a C₃₋₈cycloalkyl, a 4- to 12-membered heterocyclyl, a 6- to 12-membered aryl, or a 5- to 12-membered heteroaryl, wherein G^{1a} and G^{2a} are independently optionally substituted with 1-5 substituents independently selected from the group consisting of halogen, oxo, C₁₋₄alkyl, —OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Y¹ and Y², at each occurrence, are independently —OC₁₋₄alkyl, —OC₁₋₄haloalkyl, OH, NH₂, —NHC₁₋₄alkyl), —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
R⁵, at each occurrence, is independently halogen, cyano, oxo, C₁₋₄alkyl, C₁₋₄haloalkyl, —OR^{5a}, or C₃₋₄cycloalkyl,
R^{5a}, at each occurrence, is independently hydrogen, C₁₋₄alkyl, C₁₋₄haloalkyl, C₃₋₄cycloalkyl, or —C₁₋₃alkylene-C₃₋₄cycloalkyl; and
n is 0, 1, 2, 3, 4, or 5;
provided the compound is not 2,8,9-trimethyl-5-[4-(phenylsulfonyl)cyclohexyl]thieno[3,2-e][1,2,4]triazolo[1,5-c]pyrimidine.

E2. The compound of E1, or a pharmaceutically acceptable salt thereof, wherein the ring system of the optionally substituted 9- to 12-membered heteroaryl at G¹ is a 9-membered fused bicyclic heteroaryl having four double bonds and two to four nitrogen ring atoms, wherein one nitrogen atom occupies a position at the ring junction of the bicyclic ring system. G¹ being attached at a first carbon atom of G¹, wherein the first carbon atom of G¹ is in a 6-membered ring of the 9-membered fused bicyclic ring system

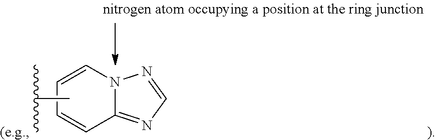

E3. The compound of E2, or a pharmaceutically acceptable salt thereof, wherein at G′ the first carbon atom and the ring junction nitrogen atom are separated by one ring

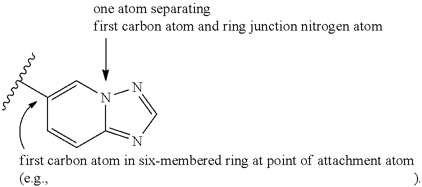 [SMILES: *c1ccc2ncnn2c1]

E4. The compound of E3, or a pharmaceutically acceptable salt thereof, wherein the ring system of the optionally substituted 9- to 12-membered heteroaryl at G¹ has the following ring system:

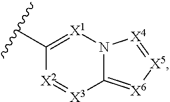

wherein x¹-x⁶ independently represent carbon or nitrogen ring atoms, provided that 1-3 of x¹-x⁶ are nitrogen atoms.

E5. The compound of E4, or a pharmaceutically acceptable salt thereof, wherein 2 x¹-x⁶ are nitrogen atoms.

E6. The compound of E5, or a pharmaceutically acceptable salt thereof, wherein the ring system

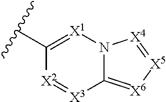

is the ring system

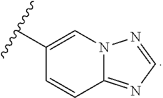 [SMILES: *c1ccc2ncnn2c1]

E7. The compound of any of E1-E4, or a pharmaceutically acceptable salt thereof, wherein G¹ is

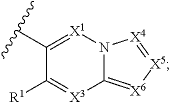

x¹, x³, x⁴, x⁵, and -x⁶ are N or CH, wherein 1-3 of x¹, x³, x⁴, x⁵, and x⁶ are N;
R¹ is C₁₋₄alkyl, C₁₋₄haloalkyl, halogen, C₂₋₄alkenyl, —OC₁₋₄alkyl, —OC₁₋₄fluoroalkyl —C(O)OR^{1a}, —C(O)NR^{1a}R^{1b}, —C₁₋₃alkylene-OH, or G^{1a};
R^{1a} and R^{1b} are each independently hydrogen or C₁₋₄alkyl; and
G^{1a} is a C₃₋₄cycloalkyl.

E7.1. The compound of E7, or a pharmaceutically acceptable salt thereof, wherein 2 of x¹, x³, x⁴, x⁵, and x⁶ are N.

E7.2. The compound of E7 or E7.1, or a pharmaceutically acceptable salt thereof, wherein R¹ is methyl, ethyl, difluoromethyl, trifluoromethyl, fluoro, chloro, methoxy, trifluoromethoxy, or cyclopropyl.

E8. The compound of E7 or E7.1, or a pharmaceutically acceptable salt thereof, wherein R¹ is C₁₋₄alkyl or halogen.

E9. The compound of E8, or a pharmaceutically acceptable salt thereof, wherein R¹ is methyl, fluoro, or chloro.

E10. The compound of any of E7-E9, or a pharmaceutically acceptable salt thereof, wherein G¹ is

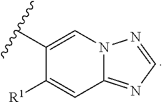

E11. The compound of any of E1-E10, or a pharmaceutically acceptable salt thereof, wherein G¹ is

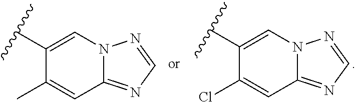 [SMILES: *c1cn2ncnc2cc1C ; *c1cn2ncnc2cc1Cl]

E12. The compound of any of E1-E11, or a pharmaceutically acceptable salt thereof, wherein G² is the optionally substituted 5- to 12 membered heteroaryl.

E13. The compound of E12, or a pharmaceutically acceptable salt thereof, wherein the ring system of the optionally substituted 5- to 12-membered heteroaryl of G² is a 5-to 6-membered monocyclic heteroaryl ring system.

E13.1. The compound of E13, or a pharmaceutically acceptable salt thereof, wherein the ring system of the optionally substituted 5- to 12-membered heteroaryl of G² is a 5-membered heteroaryl containing 1-3 ring heteroatoms independently selected from nitrogen, oxygen, and sulfur.

E13,2. The compound of E13,1, or a pharmaceutically acceptable salt thereof, wherein the ring system of the optionally substituted 5- to 12-membered heteroaryl of G² is a triazolyl, imidazolyl, thiadiazolyl, or oxadiazolyl.

E13.3, The compound of E13.2, or a pharmaceutically acceptable salt thereof, wherein the ring system of the optionally substituted 5- to 12-membered heteroaryl of G² is a triazolyl, thiadiazolyl, or oxadiazolyl.

E14. The compound of any of E13-E13.3, or a pharmaceutically acceptable salt thereof, wherein the ring system of the optionally substituted 5- to 12-membered heteroaryl of G² is 1,2,4-triazolyl, 1,3,4-thiadiazolyl, or 1,3,4-oxadiazolyl.

E14.1. The compound of any of E1-E14, or a pharmaceutically acceptable salt thereof, wherein G² is optionally substituted with 1-2 C₁₋₄alkyl.

E14.2. The compound of E14.1, or a pharmaceutically acceptable salt thereof, wherein G² is optionally substituted with 1-2 methyl.

E15. The compound of any of E12-E14.2, or a pharmaceutically acceptable salt thereof, wherein G² is

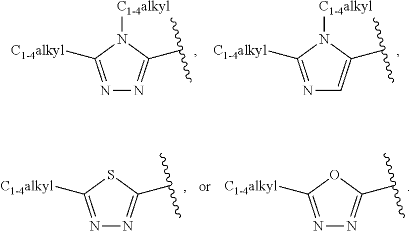

E15.1. The compound of E15, or a pharmaceutically acceptable salt thereof, wherein G² is

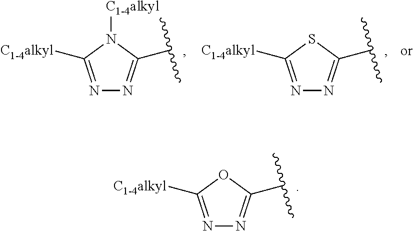

E15.2. The compound of E15 or E15.1, or a pharmaceutically acceptable salt thereof, wherein G² is

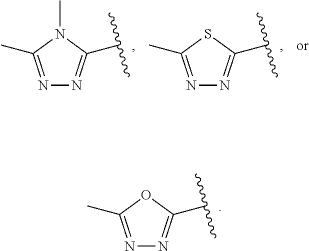

E16. The compound of any of E1-E15.2, or a pharmaceutically acceptable salt thereof, wherein L¹ is SO₂.

E17. The compound of any of E1-E16, or a pharmaceutically acceptable salt thereof, wherein n is 0.

E18. The compound of any of E1-E17, wherein “ [symbol] ” is a single bond.

E19. The compound of any of E1-E18, or a pharmaceutically acceptable salt thereof, wherein in is 1.

E20. The compound of any of E1-E19, or a pharmaceutically acceptable salt thereof, wherein the compound of formula (I) has formula (I-A):

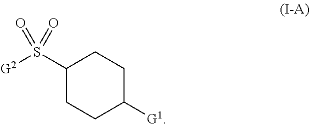

E21. The compound of E20, or a pharmaceutically acceptable salt thereof, wherein formula (I-A) has trans stereochemistry, i.e., formula (I-A1)

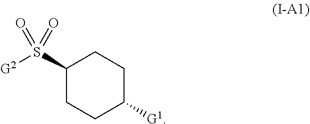

E22, The compound of E1, selected from the group consisting of:

trans-2-((4-(7-chloro-[1,2,4]triazolo[1,5-a]pyridin-6-
yl)cyclohexyl)sulfonyl)-5-methyl-1,3,4-thiadiazole;
trans-2-((4-(7-chloro-[1,2,4]triazolo[1,5-a]pyridin-6-
yl)cyclohexyl)sulfonyl)-5-methyl-1,3,4-oxadiazole;
trans-7-chloro-6-(4-((4,5-dimethyl-4H-1,2,4-triazol-3-
yl)sulfonyl)cyclohexyl)-[1,2,4]triazolo[1,5-a]pyridine;
cis-2-methyl-5-((4-(7-methyl-[1,2,4]triazolo[1,5-a]pyridin-6-
yl)cyclohexyl)sulfonyl)-1,3,4-oxadiazole;
or a pharmaceutically acceptable salt thereof.

E23. A pharmaceutical composition comprising the compound of any of E1-E22, or a pharmaceutically acceptable salt thereof, and a pharmaceutically acceptable carrier.

E24. A method of treating a psychiatric disorder comprising administering to a subject in need thereof, a therapeutically effective amount of the compound of any of E1-E22, or a pharmaceutically acceptable salt thereof, or the pharmaceutical composition of E23.

E25. The method of E24, wherein the psychiatric disorder is selected from the group consisting of substance-related disorders, opioid-related disorders, alcohol-related disorders, sedative-, hypnotic-, or anxiolytic-related disorders, stimulant-related disorders, cannabis-related disorders, hallucinogen-related disorders, inhalant-related disorders, tobacco-related disorders, depressive disorders, persistent depressive disorder (dysthymia), anxiety disorders, schizophrenia, psychotic disorder NOS, brief psychotic disorder, schizophreniform disorder, schizoaffective disorder, delusional disorder, shared psychotic disorder, catastrophic schizophrenia, postpartum psychosis, psychotic depression, psychotic break, tardive psychosis, myxedematous psychosis, occupational psychosis, menstrual psychosis, secondary psychotic disorder, bipolar I disorder with psychotic features, and substance-induced psychotic disorder.

E26. A method of inhibiting mAChR M₅ comprising administering to a subject in need thereof, a therapeutically effective amount of the compound of any of E1-E22, or a phannaceeutically acceptable salt thereof, or the pharmaceutical composition of E23.

Compound names and/or structures can be assigned/determined by using the Struct=Name naming algorithm as part of CHEMDRAW® ULTRA.

The compound may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry,” 5th edition (1989), Longman Scientific & Technical, Essex CM_{20 2}JE, England, or (2) direct separation of the mixture of optical enantiomers on chiral chromatographic columns, or (3) fractional recrystallization methods.

The present disclosure also includes an isotopically-labeled compound, which is identical to those recited in formula (1), but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium, i.e. ²H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in compounds of formula (I) are ¹¹C, ¹³N, ¹⁶O, and ¹⁸F. Isotopically-labeled compounds of formula (I) can generally be prepared by conventional techniques known to those skilled in the art or by processes analogous to those described in the accompanying Examples using appropriate isotopically-labeled reagent in place of non-isotopically-labeled reagent.

In the compounds of formula (I), any “hydrogen” or “H,” whether explicitly recited or implicit in the structure, encompasses hydrogen isotopes ¹H (protium) and ²H (deuterium). In some embodiments, hydrogen is present in its natural abundance. In some embodiments, compounds of the invention are deuterium labeled.

### a. Pharmaceutically Acceptable Salts

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quatemized with alkyl chlorides, bromides and iodides such as methyl, ethyl, propyl, isopropyl, butyl, lauryl, inyristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### b. General Synthesis

Compounds of formula (I) may be prepared by synthetic processes or by metabolic processes. Preparation of the compounds by metabolic processes includes those occurring in the human or animal body (in vivo) or processes occurring in vitro.

Abbreviations: Boc is tert-butyloxycarbonyl; Deoxo-Fluor® is bis(2-methoxyethyl)aminosulfur trifluoride; DCM is dichloromethane; DMF is N,N-dimethylformamide; DMS is dimethylsulfide; m-CPBA is meta-chloroperoxybenzoic acid; MeOH is methanol; Pd(dppf)Cl₂ is [1,1′-Bis(diphenylphosphino)ferrocene]dichloropalladium(II); PPh₃ is triphenylphosphine; TFA is trifluoroacetic acid; tosyl is 4-tolunesulfonyl.

Compounds of formula (I) may be synthesized as shown in the following schemes.

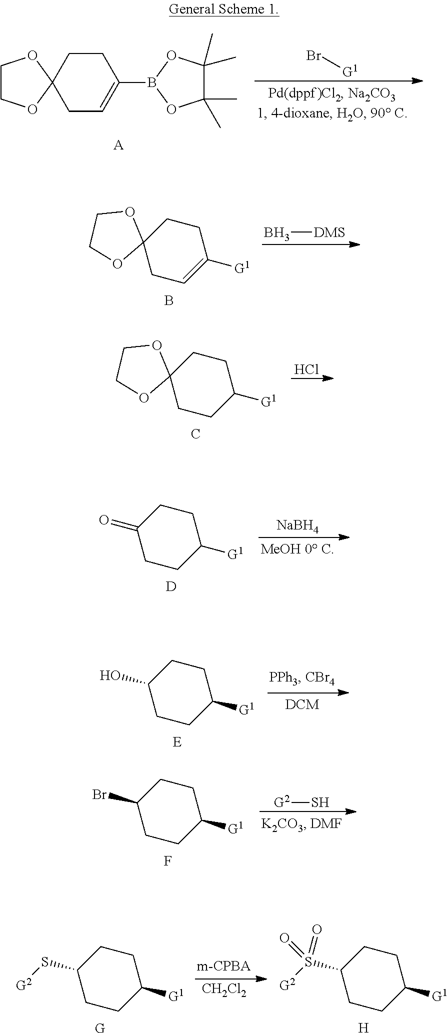

General Scheme 1 illustrates a synthetic route to provide compounds H. A boronic ester A may be coupled with Br-G¹ under Suzuki conditions to provide intermediate B, which can be reduced with borane to provide C and then deprotected to form D. Intermediate D may be reduced to intermediate E, which may be converted to the bromo intermediate F, reacted with G-SH to provide G and oxidized to compounds H.

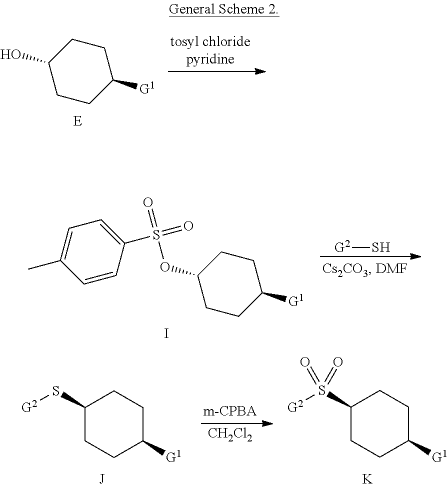

General Scheme 2 illustrates a synthetic route to provide compounds K. Intermediate E may be tosylated to form I, which may be reacted with G²-SH to provide and oxidized to compounds K.

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry,” 5th edition (1989), by Furniss, Hannaford, Smith, and Tatchell, pub. Longman Scientific &. Technical, Essex CM_{20 21}E, England.

A disclosed compound may have at least one basic nitrogen whereby the compound can be treated with an acid to form a desired salt, For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, aleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Reaction conditions and reaction times for each individual step can vary depending on the particular reactants employed and substituents present in the reactants used. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in P G M Wuts and T W Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### 3. PHARMACEUTICAL COMPOSITIONS

The compounds of the invention may be incorporated into pharmaceutical compositions suitable for administration to a subject (such as a patient, which may be a human or non-human). The compounds of the invention may also be provided as formulations, such as spray-dried dispersion formulations.

The pharmaceutical compositions may include a “therapeutically effective amount” or a “prophylactically effective amount” of the agent. A “therapeutically effective amount” refers to an amount effective, at single or multiple dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention (e.g., a compound of formula (I) or a pharmaceutically acceptable salt thereof) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds of the invention may be formulated for administration by, for example, solid dosing, eye drop, in a topical oil-based formulation, injection, inhalation (either through the mouth or the nose), implants, or oral, buccal, parenteral, or rectal administration. Techniques and formulations may generally be found in “Remington's Pharmaceutical Sciences,” (Meade Publishing Co., Easton, Pa.). Therapeutic compositions must typically be sterile and stable under the conditions of manufacture and storage.

The route by which the compounds of the invention are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90 weight % of the total composition weight.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10% of the total composition weight.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50% of the total composition weight.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10% of the total composition weight.

Suitable colorants include a colorant such as an FD&C dye. When used, the amount of colorant in a systemic or topical composition is typically about 0.005 to about 0.1% of the total composition weight.

Suitable flavors include menthol, peppermint, and fruit flavors. The amount of flavor(s), when used, in a systemic or topical composition is typically about 0.1 to about 1.0% of the total composition weight.

Suitable sweeteners include aspartame and saccharin. The amount of sweetener(s) in a systemic or topical composition is typically about 0.001 to about 1% of the total composition weight.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5% of the total composition weight.

Suitable preservatives include benzalkonium chloride, methyl paraben and sodium benzoate. The amount of presmative(s) in a systemic or topical composition is typically about 0.01 to about 5% of the total composition weight.

Suitable glidants include silicon dioxide. The amount of glidant(s) in a systemic or topical composition is typically about 1 to about 5% of the total composition weight.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100% of the total composition weight.

Suitable suspending agents include AVICEL RC-591 (from FMC Corporation of Philadelphia, PA) and sodium alginate. The amount of suspending agent(s) in a systemic or topical composition is typically about 1 to about 8% of the total composition weight.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic ingredient Handbook, 1992, pp. 587-592, Remington's Pharmaceutical Sciences, 22th Ed. 2013; and McCutcheon's Volume 1, Emulsifiers & Detergents, 1994, North American Edition, pp. 236-239. The amount of surfactant(s) in the systemic or topical composition is typically about 0.1% to about 5% of the total composition weight.

Although the amounts of components in the systemic compositions may vary depending on the type of systemic composition prepared, in general, systemic compositions include 0.01 to 50 weight % of the total composition weight of an active compound (e.g., a compound of formula (I) or a pharmaceutically acceptable salt thereof) and 50 to 99.99 weight % of the total composition weight of one or more carriers. Compositions for parenteral administration typically include 0.1 to 10 weight % of the total composition weight of actives and 90 to 99.9 weight % of the total composition weight of a carrier including a diluent and a solvent.

Compositions for oral administration can have various dosage forms. For example, solid forms include tablets, capsules, granules, and bulk powders. These oral dosage forms include a safe and effective amount, usually at least about 5 weight % of the total composition weight, and more particularly from about 25 to about 50 weight % of the total composition weight of actives. The oral dosage compositions include about 50 to about 95 weight % of carriers of the total composition weight, and more particularly, from about 50 to about 75 weight of the total composition weight.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., a compound of formula (I) or a), and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, ELDRAGIT® coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The compounds of the invention can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I) or a pharmaceutically acceptable salt thereof), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5 to about 95 weight % of the total composition weight.

Suitable propellants include propane, butane, isobutane, dimethyl ether, carbon dioxide, nitrous oxide, and combinations thereof. The amount of propellant(s) in a topical composition is typically about 0 to about 95 weight % of the total composition weight.

Suitable solvents include water, ethyl alcohol, methylene chloride, isopropanol, castor oil, ethylene glycol monoethyl ether, diethylene glycol monohutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0 to about 95 weight % of the total composition weight.

Suitable humectants include glycerin, sorbitol, sodium 2-pyrrolidone-5-carboxylate, soluble collagen, dibutyl phthalate, gelatin, and combinations thereof. Specific humectants include glycerin. The amount of hurnectant(s) in a topical composition is typically 0 to 95 weight % of the total composition weight.

The amount of thickener(s) in a topical composition is typically about 0 to about 95 weight % of the total composition weight.

Suitable powders include beta-cyclodextrins, hydroxypropyl cyclodextrins, chalk, talc, fullers earth, kaolin, starch, gums, colloidal silicon dioxide, sodium polyaciylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0 to 95 weight % of the total composition weight.

The amount of fragrance in a topical composition is typically about 0 to about 0.5 weight %, particularly, about 0.001 to about 0.1 weight % of the total composition weight.

Suitable pH adjusting additives include HCl or NaOH in amounts sufficient to adjust the pH of a topical pharmaceutical composition.

### 4. METHODS OF TREATMENT

The disclosed compounds, pharmaceutical compositions and formulations may be used in methods for treatment of disorders, such as psychiatric disorders, associated with muscarinic acetylcholine receptor dysfunction. The disclosed compounds and pharmaceutical compositions may also be used in methods for the antagonism of muscarinic acetylcholine receptor activity in a mammal, and in methods for prevention and/or treatment of substance use disorders (SUDs) in a mammal. The methods further include cotherapeutic methods for improving treatment outcomes in the context of cognitive or behavioral therapy. In the methods of use described herein, additional therapeutic agent(s) may be administered simultaneously or sequentially with the disclosed compounds and composition.

### a. Treating Disorders

The disclosed compounds, pharmaceutical compositions and formulations may be used in methods for treatment of disorders, such as psychiatric and neurological disorders, associated with muscarinic acetylcholine receptor dysfunction, or changes in DA neuron signaling that can be modulated by inhibiting M₅ activity. The methods of treatment may comprise administering to a subject in need of such treatment a therapeutically effective amount of the compound of formula (I), or a pharmaceutical composition comprising a therapeutically effective amount of a compound of formula (I).

In some embodiments, the disclosure provides a method for the prevention and/or treatment of substance use disorders (SUDs) in a mammal comprising the step of administering to the mammal a therapeutically effective amount of the compound of formula (I), or a pharmaceutical composition comprising a therapeutically effective amount of a compound of formula (I).

The compounds and compositions disclosed herein may be useful for treating, preventing, ameliorating, controlling or reducing the risk of a variety of disorders associated with selective mAChR M₅ receptor inhibition. For example, a treatment can include selective mAChR M₅ receptor inhibition to an extent effective to affect cholinergic activity. A disorder can be associated with cholinergic activity, for example cholinergic hyperfunction, A disorder also may be associated with dopaminergic activity. For example dopaminergic hyperfunction as observed in the mesolimbic dopaminergic reward pathway after exposure to substances of abuse. In addition, dopaminergic hyperfunction of both the mesolimbic and the nigro-stiatal pathways can contribute to multiple other psychiatric and neurological disorders. These include psychosis associated with schizophrenia and related psychiatric disorders, psychosis associated with neurodegenerative disorders, such as Alzheimer's disease and others, obsessive compulsive disorder, Tourette syndrome, Huntington's chorea, tardive dyskinesia, L-DOPA or DA receptor agonist-induced dyskinesia, dystonia, and other hyperkinetic or repetitive movement disorders.

Thus, provided is a method of treating or preventing a disorder in a subject comprising the step of administering to the subject at least one disclosed compound or at least one disclosed pharmaceutical composition, in an amount effective to treat the disorder in the subject.

Also provided is a method for the treatment of one or more disorders associated with mAChR M₅ receptor activity in a subject comprising the step of administering to the subject a therapeutically effective amount of the compound of formula (I), or a pharmaceutical composition comprising a therapeutically effective amount of a compound of formula (I).

In some embodiments, the disclosure provides a method for the treatment of a disorder associated with muscarinic acetylcholine receptor dysfunction or dysfunction of dopaminergic signaling in the brain reward pathway in a mammal, comprising the step of administering to the mammal an effective amount of at least one disclosed compound or a pharmaceutically acceptable salt thereof, or a pharmaceutical composition comprising at least one disclosed compound or pharmaceutically acceptable salt thereof.

In some embodiments, the disclosed compounds and compositions have utility in preventing and/or treating a variety of psychiatric disorders associated with the mAChR M₅ receptor, including one or more of the following conditions or diseases: substance-related disorders, opioid-related disorders, alcohol-related disorders, sedative-, hypnotic-, or anxiolytic-related disorders, stimulant-related disorders, cannabis-related disorders, hallucinogen-related disorders, inhalant-related disorders, tobacco-related disorders, depressive disorders including major depressive disorder (single or recurrent episode; mild, moderate, severe, with psychotic features, in partial remission, in full remission, unspecified), persistent depressive disorder (dysthymia), anxiety disorders, schizophrenia, psychotic disorder NOS, brief psychotic disorder, schizophreniform disorder, schizoaffective disorder, delusional disorder, shared psychotic disorder, catastrophic schizophrenia, postpartum psychosis, psychotic depression, psychotic break, tardive psychosis, myxedematous psychosis, occupational psychosis, menstrual psychosis, secondary psychotic disorder, bipolar I disorder with psychotic features, and substance-induced psychotic disorder. In some embodiments, the psychotic disorder is a psychosis associated with an illness selected from major depressive disorder, affective disorder, bipolar disorder, electrolyte disorder, post-traumatic stress disorder.

In some embodiments, the disorder is substance-related disorders selected from substance use disorders, substance-induced disorders, alcohol use disorder, other alcohol-induced disorders, unspecified alcohol-related disorder, caffeine-related disorders, other caffeine-induced disorders, unspecified caffeine-related disorder, cannabis-related disorders, cannabis use disorder, other cannabis-induced disorders, unspecified cannabis-related disorder, hallucinogen-related disorders, phencyclidine use disorder, other hallucinogen use disorder, hallucinogen persisting perception disorder, other phencyclidine-induced disorders, other hallucinogen-induced disorders, unspecified phencyclidine-related disorder, unspecified hallucinogen-related disorder, inhalant-related disorders, inhalant use disorder, other inhalant-induced disorders, unspecified inhalant-related disorder, opioid-related disorders, opioid use disorder, other opioid-induced disorders, unspecified opioid-related disorder, sedative-, hypnotic-, or anxiolytic-related disorders, sedative, hypnotic, or anxiolytic use disorder, other sedative-, hypnotic-, or anxiolytic-induced disorders, unspecified sedative-, hypnotic-, or anxiolytic-related disorder, stimulant-related disorders, stimulant use disorder, other stimulant-induced disorders, unspecified stimulant-related disorder, tobacco-related disorders, tobacco use disorder, other tobacco-induced disorders, unspecified tobacco-related disorder, other (or unknown) substance-related disorders, other (or unknown) substance use disorder, other (or unknown) substance-induced disorders, unspecified other (or unknown) substance-related disorder, non-substance-related disorders, gambling disorder.

In some embodiments, the disorder is depressive disorders selected from disruptive mood dysregulation disorder, major depressive disorder (single or recurrent episode; mild, moderate, severe, with psychotic features, in partial remission, in full remission, unspecified), persistent depressive disorder (dysthymia), premenstrual dysphoric disorder, substance/medication-induced depressive disorder, depressive disorder due to another medical condition, other specified depressive disorder, unspecified depressive disorder, specifiers for depressive disorders. In some embodiments, the depressive disorder is due to a general medical condition and is substance-induced or drug-induced (phencyclidine, ketamine and other dissociative anesthetics, amphetamine and other psychostimulants, and cocaine).

In some embodiments, the disorder is anxiety disorders selected from The major anxiety disorder subtypes include separation anxiety disorder, selective mutism, specific phobia, social anxiety disorder (social phobia), panic disorder, panic attack specifier, agoraphobia, generalized anxiety disorder, substance/medication-induced anxiety disorder, anxiety disorder due to another medical condition, other specified anxiety disorder, unspecified anxiety disorder. In some embodiments, the anxiety disorder is due to a general medical condition and is substance-induced or drug-induced (phencyclidine, ketamine and other dissociative anesthetics, amphetamine and other psychostimulants, and cocaine).

In some embodiments, the disorder is a psychotic disorder is selected from schizophrenia, brief psychotic disorder, schizophreniform disorder, schizoaffective disorder, delusional disorder, and shared psychotic disorder. In some embodiments, the schizophrenia is selected from catastrophic schizophrenia, catatonic schizophrenia, paranoid schizophrenia, residual schizophrenia, disorganized schizophrenia, and undifferentiated schizophrenia. In some embodiments, the disorder is selected from schizoid personality disorder, schizotypal personality disorder, and paranoid personality disorder. In some embodiments, the psychotic disorder is due to a general medical condition and is substance-induced or drug-induced (phencyclidine, ketamine and other dissociative anesthetics, amphetamine and other psychostimulants, and cocaine).

In some embodiments, the present disclosure provides a method for preventing and/or treating substance-related disorders, comprising administering to a patient in need thereof an effective amount of a compound or composition of the present disclosure. As designated by the DSM-V, substance-related disorders comprises 10 separate classes of drugs: alcohol; caffeine; cannabis; hallucinogens (with separate categories for phencyclidine [or similarly acting arylcyclohexylamines] and other hallucinogens); inhalants; opioids; sedatives, hypnotics, and anxiolytics; stimulants (amphetamine-type substances, cocaine, and other stimulants); tobacco; and other (or unknown) substances. These 10 classes are not fully distinct. All drugs that are taken in excess share a common direct activation of the mesolimbic dopaminergic reward pathway that is involved in the reinforcement of drug seeking behaviors and substance abuse. Under conditions of excessive intake of all drugs, there is an intense and direct activation of this reward pathway that can result in the neglect of normal activities. Although the pharmacological mechanisms by which each class of drugs produces reward are different, drugs of abuse typically activate this reward pathway resulting in feelings of pleasure, often referred to as a “high.” As previously described in the DSM-IV, substance use disorders (SUDs) are now encompassed as part of a broader class of disorders defined in the DSM-V under substance-related disorders, that are “related to the taking of a drug of abuse (including alcohol)”. The major or minor substance-related disorders include substance use disorders, substance-induced disorders, alcohol use disorder, other alcohol-induced disorders, unspecified alcohol-related disorder, caffeine-related disorders, other caffeine-induced disorders, unspecified caffeine-related disorder, cannabis-related disorders, cannabis use disorder, other cannabis-induced disorders, unspecified cannabis-related disorder, hallucinogen-related disorders, phencyclidine use disorder, other hallucinogen use disorder, hallucinogen persisting perception disorder, other phencyclidine-induced disorders, other hallucinogen-induced disorders, unspecified phencyclidine-related disorder, unspecified hallucinogen-related disorder, inhalant-related disorders, inhalant use disorder, other inhalant-induced disorders, unspecified inhalant-related disorder, opioid-related disorders, opioid use disorder, other opioid-induced disorders, unspecified opioid-related disorder, sedative-, hypnotic-, or anxiolytic-related disorders, sedative, hypnotic, or anxiolytic use disorder, other sedative-, hypnotic-, or anxiolytic-induced disorders, unspecified sedative-, hypnotic-, or anxiolytic-related disorder, stimulant-related disorders, stimulant use disorder, other stimulant-induced disorders, unspecified stimulant-related disorder, tobacco-related disorders, tobacco use disorder, other tobacco-induced disorders, unspecified tobacco-related disorder, nicotine use disorder, other (or unknown) substance-related disorders, other (or unknown) substance use disorder, other (or unknown) substance-induced disorders, unspecified other (or unknown) substance-related disorder, non-substance-related disorders, gambling disorder. The skilled artisan will recognize that there are alternative nomenclatures, nosologies and classification systems for mental disorders, and that these systems evolve with medical and scientific progress. Thus, the term “substance-related disorders” is intended to include like disorders that are described in other diagnostic sources.

In some embodiments, the present disclosure provides a method for treating depressive disorders, comprising administering to a patient in need thereof an effective amount of a compound or composition of the present disclosure. The fifth edition of the Diagnostic and Statistical Manual of Mental Disorders (DSM-V) (2013, American Psychiatric Association, Washington D.C.) provides a diagnostic tool for “Depressive Disorders” including disorders that share features of the presence of sad, empty, or irritable mood, accompanied by somatic and cognitive changes that significantly affect the individual's capacity to function. Differentiation of different subtypes of depressive disorders is based on the magnitude of duration, timing, or presumed etiology. In contrast with the DSM-IV, “Depressive Disorders” have been separated from “Bipolar and Related Disorders.” The major depressive disorder subtypes include disruptive mood dysregulation disorder, major depressive disorder, persistent depressive disorder (dysthymia), premenstrual dysphoric disorder, substance/medication-induced depressive disorder, depressive disorder due to another medical condition, other specified depressive disorder, unspecified depressive disorder, specifiers for depressive disorders. The skilled artisan will recognize that there are alternative nomenclatures, nosologies and classification systems for mental disorders, and that these systems evolve with medical and scientific progress. Thus the term “depressive disorders” is intended to include like disorders that are described in other diagnostic sources.

In some embodiments, the present disclosure provides a method for treating anxiety disorders, comprising administering to a patient in need thereof an effective amount of a compound or composition of the present disclosure. The fifth edition of the Diagnostic and Statistical Manual of Mental Disorders (DSM-V) (2013, American Psychiatric Association, Washington D.C.) provides a diagnostic tool for anxiety disorders including disorders that share features of excessive fear and anxiety and related behavioral disturbances. Panic attacks feature prominently within the anxiety disorders as a type of fear response. Panic attacks are not limited to anxiety disorders but rather can be observed in other mental disorders. The major anxiety disorder subtypes include separation anxiety disorder, selective mutism, specific phobia, social anxiety disorder (social phobia), panic disorder, panic attack specifier, agoraphobia, generalized anxiety disorder, substance/medication-induced anxiety disorder, anxiety disorder due to another medical condition, other specified anxiety disorder, unspecified anxiety disorder. The skilled artisan will recognize that there are alternative nomenclatures, nosologies and classification systems for mental disorders, and that these systems evolve with medical and scientific progress. Thus the term “anxiety disorders” is intended to include like disorders that are described in other diagnostic sources.

In some embodiments, the present disclosure provides a method for treating schizophrenia or psychosis, comprising administering to a patient in need thereof an effective amount of a compound or composition of the present disclosure. Particular schizophrenia or psychosis pathologies are paranoid, disorganized, catatonic or undifferentiated schizophrenia and substance-induced psychotic disorder. DSM-IV-TR provides a diagnostic tool that includes paranoid, disorganized, catatonic, undifferentiated or residual schizophrenia, and substance-induced psychotic disorder. DSM-V eliminated the subtypes of schizophrenia, and instead includes a dimensional approach to rating severity for the core symptoms of schizophrenia, to capture the heterogeneity in symptom type and severity expressed across individuals with psychotic disorders. As used herein, the term “schizophrenia or psychosis” includes treatment of those mental disorders as described in DSM-IV-TR, or DSM-V. The skilled artisan will recognize that there are alternative nomenclatures, nosologies and classification sys-tons for mental disorders, and that these systems evolve with medical arid scientific progress. Thus the term “schizophrenia or psychosis” is intended to include like disorders that are described in other diagnostic sources.

The compounds and compositions may be further useful in a method for the prevention, treatment, control, amelioration, or reduction of risk of the diseases, disorders and conditions noted herein. The compounds and compositions may be further useful in a method for the prevention, treatment, control, amelioration, or reduction of risk of the aforementioned diseases, disorders and conditions, in combination with other agents.

In the treatment of conditions Which require inhibition of mAChR M₅, an appropriate dosage level may be about 0.01 to 500 mg per kg patient body weight per day, which can be administered in single or multiple doses. The dosage level may be about 0.1 to about 250 mg/kg per day, or about 0.5 to about 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5 or 5 to 50 mg/kg per day, For oral administration, the compositions may be provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, or 1000 milligrams of the active ingredient for the symptomatic adjustment of the dosage to the patient to be treated. The compounds can be administered on a regimen of 1 to 4 times per day, preferably once or twice per day. This dosage regimen can be adjusted to provide the optimal therapeutic response. It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Thus, in some embodiments, the disclosure relates to a method for inhibiting mAChR M₅ receptor activity in at least one cell, comprising the step of contacting the at least one cell with at least one disclosed compound or at least one product of a disclosed method in an amount effective to activate mAChR M₅ in the at least one cell. In some embodiments, the cell is mammalian, for example, human. In some embodiments, the cell has been isolated from a subject prior to the contacting step. In some embodiments, contacting is via, administration to a subject.

In some embodiments, the invention relates to a method for inhibiting mAChR M₅ activity in a subject, comprising the step of administering to the subject at least one disclosed compound or at least one product of a disclosed method in a dosage and amount effective to inhibiting mAChR M₅ activity in the subject. In some embodiments, the subject is mammalian, for example, human. In some embodiments, the mammal has been diagnosed with a need for mAChR M₅ antagonism prior to the administering step. In some embodiments, the mammal has been diagnosed with a need for mAChR M₅ activation prior to the administering step. In some embodiments, the method further comprises the step of identifying a subject in need of mAChR M₅ antagonism.

In some embodiments, the invention relates to a method for the treatment of a disorder associated with selective mAChR M₅ inhibition, for example, a psychiatric disorder associated with the brain reward system, in a mammal comprising the step of administering to the mammal at least one disclosed compound or at least one product of a disclosed method in a dosage and amount effective to treat the disorder in the mammal. In some embodiments, the mammal is a human. In some embodiments, the mammal has been diagnosed with a need for treatment for the disorder prior to the administering step. In some embodiments, the method further comprises the step of identifying a subject in need of treatment for the disorder.

In some embodiments, the disorder can be selected from substance related disorders, substance use disorders, substance-induced disorders, alcohol use disorder, other alcohol-induced disorders, unspecified alcohol-related disorder, opioid-related disorders, opioid use disorder, other opioid-induced disorders, unspecified opioid-related disorder, stimulant-related disorders, stimulant use disorder, other stimulant-induced disorders, unspecified stimulant-related disorder, tobacco-related disorders, tobacco use disorder, other tobacco-induced disorders, unspecified tobacco-related disorder, other (or unknown) substance-related disorders, other (or unknown) substance use disorder, other (or unknown) substance-induced disorders, unspecified other (or unknown) substance-related disorder, non-substance-related disorders, substance related disorders associate with anxiety, substance related disorders associated with depressive disorders, substance related disorders associated with schizophrenia or psychosis.

In some embodiments, the disorder can be selected from depressive disorders, disruptive mood dysregulation disorder, major depressive disorder, persistent depressive disorder (dysthymia), premenstrual dysphoric disorder, substance/medication-induced depressive disorder, depression associated with substance-related disorders.

In some embodiments, the disorder can be selected from psychosis, schizophrenia, conduct disorder, disruptive behavior disorder, bipolar disorder, psychotic episodes of anxiety, anxiety associated with psychosis, psychotic mood disorders such as severe major depressive disorder; mood disorders associated with psychotic disorders, acute mania, depression associated with bipolar disorder, mood disorders associated with schizophrenia.

### b. Inhibition of Muscarinic Acetylcholine Receptor Activity

Compounds of the invention may pharmacologically modulate the M₅ receptor by classical antagonism of the M₅ receptor, by negative allosteric modulation of the M₅ receptor or through inverse agonism, i.e., blocking constitutively active M₅ receptors.

In some embodiments, the disclosure relates to a method for inhibition of muscarinic acetylcholine receptor activity in a mammal comprising the step of administering to the mammal an effective amount of at least one disclosed compound or a pharmaceutically acceptable salt thereof, or a pharmaceutical composition comprising at least one disclosed compound or pharmaceutically acceptable salt thereof.

In some embodiments, inhibition of muscarinic acetylcholine receptor activity decreases muscarinic acetylcholine receptor activity, decreases in brain reward system, and/or decreases mesolimbic dopamine reward pathway activity. In some embodiments, inhibition of muscarinic acetylcholine receptor activity is partial antagonism of the muscarinic acetylcholine receptor. In some embodiments, inhibition of muscarinic acetylcholine receptor activity is negative allosteric modulation of the muscarinic acetylcholine receptor.

In an embodiment, a compound of the invention inhibits the agonist response (e.g., acetylcholine) of mAChR M₅. In some embodiments, a compound of the invention decreases mAChR M₅ response to a near maximal concentration of an agonist (e.g, an EC₈₀ of Ach)) in the presence of compound of the invention. The inhibition of mAChR M₅ activity can be demonstrated by methodology known in the art. For example, activation of mAChR M₅ activity can be determined by measurement of calcium flux in response to an agonist, e.g, acetylcholine, in cells loaded with a Ca²⁺-sensitive fluorescent dye (e.g., Fluo-4). In an embodiment, the calcium flux was measured as an increase in fluorescent static ratio. In an embodiment, competitive and non-competitive antagonist activity was analyzed as a concentration-dependent decrease in the EC₈₀ acetylcholine response (i.e. the response of mAChR M₅ at a concentration of acetylcholine that yields 80% of the maximal response).

In an embodiment, a compound of the invention inhibits mAChR M₅ response as a decrease in calcium fluorescence in mAChR M₅-transfected CHO-K1 cells in the presence of a compound of the invention.

The compounds of the invention may exhibit competitive and non-competitive antagonism of mAChR M₅ response to acetylcholine as a decrease in response to non-maximal concentrations of acetylcholine in CHO-K1 cells transfected with a mAChR M₅ in the presence of the compound, compared to the response to acetylcholine in the absence of the compound.

In some embodiments, the compound administered exhibits inhibition of mAChR M₅ with an IC₅₀ of less than about 10 μM, less than about 5 μM, less than about 1 μM, less than about 500 nM, or less than about 100 nM. In some embodiments, the compound administered exhibits inhibition of mAChR M₅ with an IC₅₀ of between about 10 μM and about 1 nM, about 1 μM and about 1 nM, about 100 nM and about 1 nM, or about 10 nM and about 1 nM.

In some embodiments, the mammal is a human. In some embodiments, the mammal has been diagnosed with a need for inhibition of muscarinic acetylcholine receptor activity prior to the administering step. In some embodiments, the method further comprises the step of identifying a mammal in need of inhibiting muscarinic acetylcholine receptor activity. In some embodiments, the inhibition of muscarinic acetylcholine receptor activity treats a disorder associated with muscarinic acetylcholine receptor activity in the mammal.

In some embodiments, the inhibition of muscarinic acetylcholine receptor activity prevents a disorder associated with muscarinic acetylcholine receptor activity in the mammal. In some embodiments, the muscarinic acetylcholine receptor is mAChR M₅.

In some embodiments, the mammal is a human. In some embodiments, the mammal has been diagnosed with a need for inhibition of muscarinic acetylcholine receptor activity prior to the administering step. In some embodiments, the method further comprises the step of identifying a mammal in need of inhibiting muscarinic acetylcholine receptor activity. In some embodiments, the inhibition of muscarinic acetylcholine receptor activity treats a psychiatric disorder associated with brain reward system in the mammal. In some embodiments, the inhibition of muscarinic acetylcholine receptor activity prevents a psychiatric disorder associated with brain reward system in the mammal. In some embodiments, the muscarinic acetylcholine receptor is mAChR M₅.

In some embodiments, inhibition of muscarinic acetylcholine receptor activity in a mammal is associated with the treatment of a psychiatric disorder associated with a muscarinic receptor dysfunction, such as a neurological or psychiatric disorder disclosed herein. In some embodiments, the muscarinic receptor is mAChR M₅.

In some embodiments, inhibition of muscarinic acetylcholine receptor activity in a mammal is associated with the treatment of a psychiatric disorder associated with brain reward system, such as a psychiatric disorder disclosed herein. In some embodiments, the muscarinic receptor is mAChR M₅.

In some embodiments, inhibition of muscarinic acetylcholine receptor activity in a mammal is associated with the prevention of a psychiatric disorder associated with brain reward system, such as a psychiatric disorder disclosed herein. In some embodiments, the muscarinic receptor is mAChR M₅.

In some embodiments, the disclosure provides a method for inhibition of muscarinic acetylcholine receptor activity in a cell, comprising the step of contacting the cell with an effective amount of at least one disclosed compound or a pharmaceutically acceptable salt thereof. In some embodiments, the cell is mammalian (e.g., human). In some embodiments, the cell has been isolated from a mammal prior to the contacting step. In some embodiments, contacting is via administration to a mammal.

In vivo efficacy for compounds of the invention may be measured in a number of preclinical behavioral models Efficacy may be measured by reversal of oxycodone self-administration or inhibition of cue-induced relapse of oxycodone drug seeking behavior in mammals after forced abstinence, referred to as reversal of cue-induced reactivity (Gould et al. ACS Chem Neurosci (2019) 10: 3740-37502019). Compounds of the invention may reverse the locomotor hyperactivity response induced by systemic administration of an acute dose of oxycodone, referred to as reversal of oxycodone-induced hyperactivity.

### c. Inhibition of Substance-Related Misuse

In some embodiments, the invention relates to a method for prevention of substance-related misuse in a mammal comprising the step of administering to the mammal an effective amount of least one disclosed compound; or a pharmaceutically acceptable salt, hydrate, solvate, or polymorph thereof. In some embodiments, the mammal is a human. In some embodiments, the method comprises the step of preventing in a mammal substance-related misuse. In some embodiments, the need for substance-related misuse prevention is associated with a muscarinic receptor dysfunction. In some embodiments, the muscarinic receptor is mAChR M₅. In some embodiments, the need for substance-related misuse prevention is associated with dysfunction of the brain reward system including the mesolimbic dopamine reward pathway.

In some embodiments, the invention relates to a method for prevention of opioid-related misuse in a mammal comprising the step of administering to the mammal an effective amount of least one disclosed compound; or a pharmaceutically acceptable salt, hydrate, solvate, or polymorph thereof. In some embodiments, the mammal is a human. In some embodiments, the method comprises the step of preventing in a mammal opioid-related misuse. In some embodiments, the need for opioid-related misuse prevention is associated with a muscarinic receptor dysfunction. In some embodiments, the need for opioid-related misuse prevention is associated with dysfunction of the brain reward system including the mesolimbic dopamine reward pathway. In some embodiments, the muscarinic receptor is mAChR M₅.

In some embodiments, the prevention of opioid-related misuse is a statistically significant prevention of opioid self-administration in rodents. In some embodiments, the prevention of opioid-related misuse is a statistically significant decreased opioid misuse in the Drug Use Screening Inventory-Revised (DUSI-R).

### d. Inhibition of Substance-Related Disorder Relapse

In some embodiments, the invention relates to a method for inhibiting relapse of substance-related disorder in a mammal comprising the step of administering to the mammal an effective amount of least one disclosed compound; or a pharmaceutically acceptable salt, hydrate, solvate, or polymorph thereof. In some embodiments, the mammal is a human. In some embodiments, the mammal has been diagnosed with a need for inhibition of substance-related disorder prior to the administering step. In some embodiments, the method further comprises the step of identifying a mammal in need of substance-related disorder inhibition. In some embodiments, the need for inhibition of substance-related disorder relapse is associated with a muscarinic receptor dysfunction. In some embodiments, the need for inhibition of substance-related disorder relapse is associated with dysfunction of the brain reward system including the mesolimbic dopamine reward pathway. In some embodiments, the muscarinic receptor is mAChR M₅.

In some embodiments, the invention relates to a method for inhibiting relapse of opioid-related disorders in a mammal comprising the step of administering to the mammal an effective amount of least one disclosed compound; or a pharmaceutically acceptable salt, hydrate, solvate, or polymorph thereof. In some embodiments, the mammal is a human. In some embodiments, the mammal has been diagnosed with a need for inhibition of opioid-related disorders prior to the administering step. In some embodiments, the method further comprises the step of identifying a mammal in need of opioid-related disorders inhibition. In some embodiments, the need for inhibition of relapse of opioid-related disorders is associated with a muscarinic receptor dysfunction. In some embodiments, the need for inhibition of relapse of opioid-related disorders is associated with dysfunction of the brain reward system including the mesolimbic dopamine reward pathway. In some embodiments, the muscarinic receptor is mAChR M₅.

In some embodiments, the inhibition of relapse of opioid-related disorders is a statistically significant decrease in opioid self-administration or cue-induced relapse of opioid self-administration. In some embodiments, the inhibition of relapse of opioid-related disorders is a statistically significant decreased opioid abuse in the Drug Use Screening Inventory-Revised (DUSI-R).

In some embodiments, the invention relates to a method for inhibiting relapse of alcohol-related disorders in a mammal comprising the step of administering to the mammal an effective amount of least one disclosed compound; or a pharmaceutically acceptable salt, hydrate, solvate, or polymorph thereof. In some embodiments, the mammal is a human. In some embodiments, the mammal has been diagnosed with a need for inhibition of alcohol-related related disorders prior to the administering step. In some embodiments, the method further comprises the step of identifying a mammal in need of alcohol-related disorders inhibition. In some embodiments, the need for inhibition of relapse of alcohol-related disorders is associated with a muscarinic receptor dysfunction. In some embodiments, the need for inhibition of relapse of alcohol-related disorders is associated with dysfunction of the brain reward system including the mesolimbic dopamine reward pathway. In some embodiments, the muscarinic receptor is mAChR M₅.

In some embodiments, the inhibition of relapse of alcohol-related disorders is a statistically significant decrease in alcohol drinking or cue-induced relapse of alcohol drinking in rodents. In some embodiments, the inhibition of relapse of alcohol-related disorders is a statistically significant decreased alcohol use in the Drug Use Screening Inventory-Revised (DUSI-R) or Adult Subsetance Use Survey (ASUS).

In some embodiments, the invention relates to a method for inhibiting relapse of tobacco-related disorders in a mammal comprising the step of administering to the mammal an effective amount of least one disclosed compound; or a pharmaceutically acceptable salt, hydrate, solvate, or polymorph thereof. In some embodiments, the mammal is a human. In some embodiments, the mammal has been diagnosed with a need for inhibition of tobacco-related disorders prior to the administering step. In some embodiments, the method further comprises the step of identifying a mammal in need of tobacco-related disorders inhibition. In some embodiments, the need for the inhibition of relapse of tobacco-related disorders is associated with a muscarinic receptor dysfunction. In some embodiments, the need for inhibitor) of relapse of tobacco-related use disorders is associated with dysfunction of the brain reward system including the mesolimbic dopamine reward pathway. In some embodiments, the muscarinic receptor is mAChR M₅.

In some embodiments, the inhibition of tobacco-related disorders is a statistically significant decrease in nicotine self-administration or cue-induced relapse of nicotine self-administration in rodents. In some embodiments, the inhibition of tobacco-related disorders is a statistically significant decreased tobacco or nicotine use in the Fagerstrom Test for Nicotine Dependence.

In some embodiments, the invention relates to a method for inhibiting relapse of cocaine-related disorders in a mammal comprising the step of administering to the mammal an effective amount of least one disclosed compound; or a pharmaceutically acceptable salt, hydrate, solvate, or polymorph thereof. In some embodiments, the mammal is a human. In some embodiments, the mammal has been diagnosed with a need for inhibition of cocaine-related disorders prior to the administering step. In some embodiments, the method further comprises the step of identifying a mammal in need of cocaine-related disorders inhibition. In some embodiments, the need for inhibition of relapse of cocaine-related disorders is associated with a muscarinic receptor dysfunction. In some embodiments, the need for inhibition of relapse of cocaine-related disorders is associated with dysfunction of the brain reward system including the mesolimbic dopamine reward pathway. In some embodiments, the muscarinic receptor is mAChR M₅.

In some embodiments, the inhibition of relapse of cocaine-related disorders is a statistically significant decrease in cocaine self-administration or cue-induced relapse of cocaine self-administration in rodents. In some embodiments, the inhibition of relapse of cocaine-related disorders is a statistically significant decreased cocaine use in the Drug Use Screening Inventory Revised (DUSI-R).

### e. Inhibition of Anxiety

In some embodiments, the invention relates to a method for inhibiting anxiety in a mammal comprising the step of administering to the mammal an effective amount of least one disclosed compound; or a pharmaceutically acceptable salt, hydrate, solvate, or polymorph thereof. In some embodiments, the mammal is a human. In some embodiments, the mammal has been diagnosed with a need for inhibition of anxiety prior to the administering step. In some embodiments, the method further comprises the step of identifying a mammal in need of anxiety inhibition. In some embodiments, the need for anxiety inhibition is associated with a muscarinic receptor dysfunction. In some embodiments, the muscarinic receptor is mAChR M₅.

In some embodiments, the inhibition of anxiety is a statistically significant increased time spent in open arm of elevated plus maze task in rodents. In some embodiments, the inhibition of anxiety is a statistically significant decrease in anxiety ratings in the Beck Anxiety Inventory (BAI).

### f. Inhibition of Depression

In some embodiments, the invention relates to a method for inhibiting depression in a mammal comprising the step of administering to the mammal an effective amount of least one disclosed compound; or a pharmaceutically acceptable salt, hydrate, solvate, or polymorph thereof. In some embodiments, the mammal is a human. In some embodiments, the mammal has been diagnosed with a need for inhibition of depression prior to the administering step. In some embodiments, the method further comprises the step of identifying a mammal in need of depression inhibition. In some embodiments, the need for depression inhibition is associated with a muscarinic receptor dysfunction. In some embodiments, the muscarinic receptor is mAChR M₅.

In some embodiments, the inhibition of depression is a statistically significant decrease in immobilization of the forced swim task or tail suspension in rodents. In some embodiments, the inhibition of psychosis is a statistically significant increase mood in Hamilton Depression Rating Scale (HAM-D).

### g. Inhibition of Psychosis

In some embodiments, the invention relates to a method for inhibiting psychosis in a mammal comprising the step of administering to the mammal an effective amount of least one disclosed compound; or a pharmaceutically acceptable salt, hydrate, solvate, or polymorph thereof. In some embodiments, the mammal is a human. In some embodiments, the mammal has been diagnosed with a need for inhibition of psychosis prior to the administering step. In some embodiments, the method further comprises the step of identifying a mammal in need of psychosis inhibition. In some embodiments, the need for psychosis inhibition is associated with a muscarinic receptor dysfunction. In some embodiments, the muscarinic receptor is mAChR M₅.

In some embodiments, the inhibition of psychosis is a statistically significant decrease in amphetamine-induced hyperactivity. In some embodiments, the inhibition of psychosis is a statistically significant decrease in the positive symptom scales of the Positive and Negative Syndrome Scale (PANSS) or Brief Psychiatric Rating Scale (BPRS

### h. Cotherapeutic Methods

In the methods of use described herein, additional therapeutic agent(s) may be administered simultaneously or sequentially with the disclosed compounds and compositions. Sequential administration includes administration before or after the disclosed compounds and compositions. In some embodiments, the additional therapeutic agent or agents may be administered in the same composition as the disclosed compounds, in other embodiments, there may be an interval of time between administration of the additional therapeutic agent and the disclosed compounds. In some embodiments, administration of an additional therapeutic agent with a disclosed compound may allow lower doses of the other therapeutic agents and/or administration at less frequent intervals. When used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula (I). The above combinations include combinations of a compound of the present invention not only with one other active compound, but also with two or more other active compounds.

The disclosed compounds can be used as single agents or in combination with one or more other drugs in the treatment, prevention, control, amelioration or reduction of risk of the aforementioned diseases, disorders and conditions for which the compound or the other drugs have utility, where the combination of drugs together are safer or more effective than either drug alone. The other drug(s) can be administered by a route and in an amount commonly used therefor, contemporaneously or sequentially with a disclosed compound. When a disclosed compound is used contemporaneously with one or more other drugs, a pharmaceutical composition in unit dosage form containing such drugs and the disclosed compound may be used. However, the combination therapy can also be administered on overlapping schedules. It is also envisioned that the combination of one or more active ingredients and a disclosed compound can be more efficacious than either as a single agent. Thus, when used in combination with one or more other active ingredients, the disclosed compounds and the other active ingredients can be used in lower doses than when each is used singly.

The pharmaceutical compositions and methods of the present invention can further comprise other therapeutically active compounds as noted herein which are usually applied in the treatment of the above-mentioned pathological conditions.

The above combinations include combinations of a disclosed compound not only with one other active compound, but also with two or more other active compounds. Likewise, disclosed compounds can be used in combination with other drugs that are used in the prevention, treatment, control, amelioration, or reduction of risk of the diseases or conditions for which disclosed compounds are useful. Such other drugs can be administered, by a route and in an amount commonly used therefor, contemporaneously or sequentially with a compound of the present invention. When a compound of the present invention is used contemporaneously with one or more other drugs, a pharmaceutical composition containing such other drugs in addition to a disclosed compound is preferred. Accordingly, the pharmaceutical compositions include those that also contain one or more other active ingredients, in addition to a compound of the present invention.

The weight ratio of a disclosed compound to the second active ingredient can be varied and will depend upon the effective dose of each ingredient. Generally, an effective dose of each will be used. Thus, for example, when a compound of the present invention is combined with another agent, the weight ratio of a disclosed compound to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations a disclosed compound and other active agents can be administered separately or in conjunction. In addition, the administration of one element can be prior to, concurrent to, or subsequent to the administration of other agent(s).

In some embodiments, the compound can be employed in combination with one or more commonly prescribed opioid analgesics for prevention of misuse or relapse including alfentanil IV; buprenorphine (buccal film, film/tablet, SubQ, patch, IV); butorophanol oral; codeine oral; dextromethorphan oral; dihydrocodeine oral; fentanyl (buccal or SL tablets, lozenge/troche, film or oral spray, nasal spray, patch, IV, epidural, intrathecal); hydrocodone oral; hydromorphone (epidural, IV, oral/rectal); levorphanol (IV and oral); loperamide (oral), meperidine (IV and oral); methadone (oral, IV); morphine (IV, epidural, intrathecal, oral/rectal); nalbuphine IV; opium oral; oxycodone oral; oxymorphone IV; oxymorphone oral; pentazocine (IV and oral); remifentanil IV; sufentanil (IV and epidural); tapentadol oral; tramadol oral.

In some embodiments, the compound can be employed alone in combination with one or more classes of drugs commonly associated with substance-related disorders for prevention of misuse or relapse, including alcohol; caffeine; cannabis; hallucinogens (with separate categories for phencyclidine [or similarly acting arylcyclohexylamines] and other hallucinogens); inhalants; opioids; sedatives, hypnotics, and anxiolytics; stimulants (amphetamine-type substances, cocaine, and other stimulants); and tobacco.

In some embodiments, the compound can be employed alone in combination with one or more classes of drugs commonly associated used for the prevention of relapse of substance-related disorders including naloxone (IV, IM, SC, endotracheal, sublingual, intralingual, submental, and nasal routes), naltrexone, acamprosate, disulfiram, topiramate gabapentin, bupriopion, bupropion/naltrexone, varenicline, nicotine replacement (gum, patch, lozenge), benzodiazepine, hormone therapy, buprenorphine (alone, combined with naloxone, monthly injection, sublingual tablets), gabapbetin, topiramate, varenicline, behavioral therapies including cognitive-behavioral therapy (CBT).

In some embodiments, the compound can be employed in combination with one or more commonly prescribed non-opioid analgesics non-opioid pain medications including NSAIDS (non-steriodal anti-inflammatory drugs) including ibuproden oral, naproxen oral, ketorolac (oral, IM, IV), diaclodenac (oral, topical gel), etodolac oral, meloxicam oral, methyl salicylate/menthol (topical); steroids (oral, intra-articular, peri-neural, epidural, IM, IV); anticonvulsants including gabapentin and pregabalin oral; SNRIs including duloxetine and milnacipran; tricycelic anti-depressants including amitriptyline, nortriptyline and desipramine; sodium channel blocker including lidocaine (topical cream/patch, IM, IV) fnexilitine, topiramate; TRPV I ion channel blocker including capsaicin (topical cream patch, ointment); NMDA antagonists including ketamine IV, memantine oral, dextromethorphan; antispasmotics including cyclobenzaprine, tizanidine, baclofen, diazepam, lorazepam; acetaminophen oral; alpha agonists including clonidine (oral, patch), dexmedetomidine IV, guanfacine oral.

In some embodiments, the compound can be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, aceto-phenazine, fluphenazine, perphenazine and trifluoperazine. Suitable examples ofthioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the subject compound can be in the form of a pharmaceutically acceptable salt, for example, chlorpromazine hydroChloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydroChloride. Perphenazine, chlorprothixene, clozapine, haloperidol, pimozide and risperidone are commonly used in a non-salt form. Thus, the subject compound can be employed in combination with acetophenazine, alentemol, aripiprazole, amisulpride, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisu-ride, loxapine, mesoridazine, molindolone, naxagolide, olanzapine, pergolide, perphenazine, pimozide, pramipexole, quetiapine, risperidone, sulpiride, tetrabenazine, trihexyphenidyl, thioridazine, thiothixene, trifluoperazine or ziprasidone.

In some embodiments, the compound can be employed in combination with an antidepressant or antianxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, alpha-adrenoreceptor antagonists, neurokinin-1 receptor antagonists, atypical antidepressants, benzodiazepines, 5-HTIA agonists or antagonists, especially 5-HTIA partial agonists, and corticotropin releasing factor (CRF) antagonists. Specific agents include: amitriptyline, clomiprainine, doxepin, imipramine and trimipramine; amoxapine, desipramine, maprotiline, nortriptyline and protriptyline; fluoxetine, fluvoxamine, paroxetine and sertraline; isocarboxazid, pheneizine, tranylcypromine and selegiline; moclobemide; venlafaxine; duloxetine; aprepitant; bupropion, nefazodone, trazodone and viloxazine; alprazolam, chlordiazepoxide, clonazepam, chlorazepate, diazepam, halazepam, lorazepam, oxazepam and prazepam; buspirone, tiesinoxan, gepirone and ipsapirone, and pharmaceutically acceptable salts thereof.

### i. Modes of Administration

Methods of treatment may include any number of modes of administering a disclosed composition. Modes of administration may include tablets, pills, dragees, hard and soft gel capsules, granules, pellets, aqueous, lipid, oily or other solutions, emulsions such as oil-in-water emulsions, liposomes, aqueous or oily suspensions, syrups, elixirs, solid emulsions, solid dispersions or dispersible powders. For the preparation of pharmaceutical compositions for oral administration, the agent may be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. Gelucire™). In the pharmaceutical composition, the agent may also be dispersed in a microparticle, e.g. a nanoparticulate composition.

For parenteral administration, the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration, the agent can be in the form of an aqueous, lipid, oily or other kind of solution or suspension or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

For transdermal administration, agents may be formulated using one of the following delivery systems application, including single-layer drug-in-adhesive in which the adhesive layer of system contains the agent or multi-layer drug-in-adhesive in which one layer acts for immediate release of the drug and other layers control release of drug from the reservoir with release dependent on membrane permeability and diffusion of drug molecules; reservoir transdermal system with separate liquid compartment containing the agent solution or suspension separated by the adhesive layer allowing with zero order release rates; and matrix systems (monolithic device) with a layer of a semisolid matrix containing an agent solution or suspension and surrounding adhesive layer.

### 5. KITS

In one aspect, the disclosure provides a kit comprising at least one disclosed compound or a pharmaceutically acceptable salt thereof, or a pharmaceutical composition comprising at least one disclosed compound or a pharmaceutically acceptable salt thereof and one or more of:
(a) at least one agent known to decrease mAChR M₅ activity;
(b) at least one agent known to treat a disorder associated with mAChR M₅, such as a disorder described herein;
(c) at least one agent known to treat a disorder associated with the brain reward system, such as a disorder described herein; and
(d) instructions for administering the compound.

In some embodiments, the at least one disclosed compound and the at least one agent are co-formulated. In some embodiments, the at least one disclosed compound and the at least one agent are co-packaged. The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

That the disclosed kits can be employed in connection with disclosed methods of use.

The kits may further comprise information, instructions, or both that use of the kit will provide treatment for medical conditions in mammals (particularly humans). The information and instructions may be in the form of words, pictures, or both, and the like. In addition or in the alternative, the kit may include the compound, a composition, or both; and information, instructions, or both, regarding methods of application of compound, or of composition, preferably with the benefit of treating or preventing medical conditions in mammals (e.g., humans).

The compounds and processes of the invention will be better understood by reference to the following examples, which are intended as an illustration of and not a limitation upon the scope of the invention.

### 6. EXAMPLES

NMR spectra were recorded on a 400 MHz AMX Balker NMR spectrometer. ¹H chemical shifts are reported in δ values in ppm downfield with the deuterated solvent as the internal standard. Data are reported as follows: chemical shift, multiplicity (s=singlet, bs=broad singlet, d=doublet, t=triplet, q=quartet, dd=doublet of doublets, m=multiplet, ABq=AB quartet), coupling constant, integration. Reversed-phase LCMS analysis was performed using an Agilent 1200 system comprised of a binary pump with degasser, high-performance autosampler, thermostatted column compartment, C18 column, diode-array detector (DAD) and an Agilent 6150 MSD with the following parameters. The gradient conditions were 5% to 95% acetonitrile with the aqueous phase 0.1% TFA in water over 1.4 minutes. Samples were separated on a Waters Acquity UPLC BEH C18 column (1.7 μm, 1.0×50 mm) at 0.5 mL/min, with column and solvent temperatures maintained at 55° C. The DAD was set to scan from 190 to 300 nm, and the signals used were 220 nm and 254 nm (both with a band width of 4 nm). The MS detector was configured with an electrospray ionization source, and the low-resolution mass spectra were acquired by scanning from 140 to 700 AMU with a step size of 0.2 AMU at 0.13 cycles/second, and peak width of 0.008 minutes. The drying gas flow was set to 13 liters per minute at 300° C. and the nebulizer pressure was set to 30 psi. The capillary needle voltage was set at 3000 V, and the fragmentor voltage was set at 100V. Data acquisition was performed with Agilent Chemstation and Analytical Studio Reviewer software.

### a. Abbreviations

atm is atmosphere(s)
Boc is tort-butyloxycarbonvi
Boc₂O is di-tert-butyl dicarbonate
DCE is 1,2-dichloroethane
DCM is dichloromethane
Deoxo-Fluor® is bis(2-methoxyethyl)aminosulfur trifluoride
DIPEA is N,N-diisopropylethylamine
DMF is N,N-dimethylformamide
DMS is dimethylsulfide
DMSO is dimethylsulfoxide
eq or equiv is equivalent(s)
EtOAc is ethyl acetate
EtOH is ethanol
Et₃N is triethylamine
HATU is 2-(7-aza-1H-benzotriazole-1 -yl)-1,1,3,3-tetramethyluronium hexafluorophosphate
h or h. is hour(s)
hex is hexane
IPA or iPA is isopropyl alcohol
m-CPBA is meta-chloroperoxybenzoic acid
LCMS is liquid chromatography mass spectrometry
MeCN is acetonitrile
MeOH is methanol
min or min. is minute(s)
NaOMe is sodium methoxide
NMP is N-methyl-2-pyrrolidone
NCS is N-Chlorosuccinimide
Pd(dppf)Cl₂ is [1,1′-Bis(diphenylphosphino)ferrocene]dichloropalladium(II)
PE is petroleum ether
PPh₃ is triphenylphosphine
RP-HPLC is reverse phase high-performance liquid chromatography
rt, RT, or Et, is room temperature
sat, is saturated
Selectfluor™ is 1-chloromethyl-4-fluoro-1,4-diazoniabicyclo[2.2.2]octane bis(tetrafluoroborate)
TFA is trifluoroacetic acid
THE is tetrahydrofuran
tosyl is 4-toluenesulfonyl

### b. Preparation of Intermediates

### Intermediate Example 1, 6-Bromo-7-chloro-[1,2,4]triazolo[1,5-a] pyridine

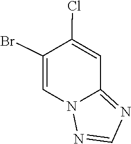 [SMILES: Clc1cc2ncnn2cc1Br]

Step A. N-(5-bromo-4-chloro-2-pyridyl)-N′-hydroxy-formamidine. 5-Bromo-4-chloro-2-aminopyridine (4 g, 19.3 mmol, 1 eq) was added to a round bottom flask. iPA (64 mL) and N,N-dimethylformamide dimethyl acetal (3.3 mL, 25.1 mmol, 1.3 eq) were added, and the resulting mixture was heated to 82° C. for 3 h, after which time the reaction was cooled to 50° C. Hydroxylamine hydrochloride (1.74 g, 25.1 mmol, 1.3 eq) was added in one portion, and the reaction was stirred at 50° C. for 2 h, after which time the reaction was cooled to room temperature and concentrated under reduced pressure to provide the crude mixture of N-(5-bromo-4-chloro-2-pyridyl)-N′-hydroxy-formamidine as a yellow solid, which was directly used without further purification.

Step B. 6-Bromo-7-chloro-[1,2,4]triazolo[1.5-a]pyridine. N-(5-Bromo-4-chloro-2-pyridyl)-N′-hydroxy-formamidine (4.83 g, 19.3 mmol, 1 eq) was added to a round bottom flask. THF (55 mL) was added, and the resulting mixture was cooled to 0° C. Trifluoroacetic anhydride (8 mL, 57.8 mmol, 3 eq) was then added by syringe, and the reaction was stirred at room temperature overnight, after which time the reaction was quenched with 1 N NaOH (55 mL), and then extracted with CHCl₃/iPA solution (3:1). The combined organic extracts were concentrated and dried over Na₂SO₄, and solvents were filtered and concentrated. The crude residue was then purified by column chromatography (0-100% EtOAc in hexanes) to give the title compound (3.93 g. 87% over 2 steps). ¹H-NMR (400 MHz, MeOD) δ 9.51 (s, 1H), 8.80 (s, 1H), 8.25 (s, 1H). ES-MS [M+H]⁺=232.2.

The compounds shown in Table 1 may be prepared similarly to the compound described above, with appropriate starting materials.

TABLE 1

| No. | Structure | Name | ¹H-NMR and/or ES-MS [M + H]⁺ |
|---|---|---|---|
| 1 | 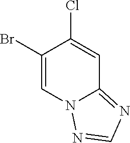 [SMILES: Clc1cc2ncnn2cc1Br] | 6-bromo-7-chloro- [1,2,4]triazolo[1,5-a]pyridine | ¹H-NMR (400 MHz, MeOD) δ 9.51 (s, 1H), 8.80 (s, 1H), 8.25 (s, 1H). ES-MS [M + H]⁺ = 232.2. |
| 2 | 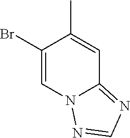 | 6-bromo-7-methyl- [1,2,4]triazolo[1,5-a]pyridine | ¹H-NMR (400 MHz, MeOD) δ 9.53 (s, 1H), 9.16 (s, 1H), 8.09 (s, 1H), 2.72 (s, 3H). ES-MS [M + H]⁺ = 212.2 and 214.3. |

### Intermediate Example 2. cis-6-(4-Bromocyclohexyl)-7-chloro-[1,2,4]triazolor[1,5-a]pyridine

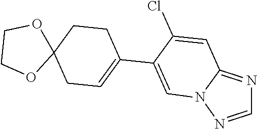

Step A. 7-Chloro-6-(1,4-dioxaspiro[4.5]dec-7-en-8-yl)-[1,2,4]triazolo[1,5-a]pyridine. To a solution of 6-bromo-7-chloro-[1,2,4]triazolo[1,5-a]pyridine (5.0 g, 21.5 mmol, 1.0 eq) its 1,4-dioxane (80 mL) and. H₂O (20 mL) was added 2-(1,4-dioxaspiro[4.5] dec-7-en-8-yl)-4,4,5,5-tetramethyl-1,3,2-dioxaborolane (6.3 g, 23.7 mmol, 1.1 eq), Na₂CO₃ (6.97 g, 64.5 mmol, 3.0 eq) and Pd(dppf)Cl₂DCM (880.4 mg, 1.08 mmol, 0.05 eq). The mixture was stirred at 90° C. for 5 h under N₂, after which time, the reaction mixture was filtered, diluted with H₂O (50 mL) and extracted with CH₂Cl₂ (50 mL×3). The combined organic layers were washed with brine (50 mL×2), dried over Na₂SO₄, filtered and concentrated under reduced pressure to give a crude residue. The crude residue was then purified by column chromatography (0-100% EtOAc in hexanes) to give the title compound (4.6 g, 73%). ¹H-NMR (400 MHz, CDCl₃) δ 8.46 (s. 1H), 8.33 (s, 1H), 7.81 (s, 1H), 5.75 (tt, J=3.8, 1.5 Hz, 1H), 1.04 (p. J=1.7 Hz, 4H), 2.58 (tq. J=6.1, 2.0 Hz, 2H), 2.49 (dt, J=4.6, 2.3 Hz, 2H), 1.93 (t, J=6.4 Hz, 2H). ES-MS [M+H]⁺=292.4.

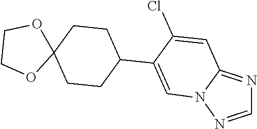

Step B. 7-Chloro-6-(1,4-dioxaspiro[4.5]decan-8-yl)-[1,2,4]triazolo[1.5-a]pyridine. To a solution of 7-chloro-6-(1,4-dioxaspiro[4.5]dec-7-en-8-yl)-[1,2,4]triazolo[1,5-a]pyridine (4.6 g, 15.8 mmol, 1.0 eq) in THF (50 mL) at 0° C. was added BH₃·DMS (48.0 mL, 96 mmol, 6.1 eq). The mixture was stirred at room temperature for 48 h under N₂. The mixture was then quenched with 3N NaOH (40 mL) at 0° C. carefully and stirred at 60° C. for 3 h. The reaction mixture was concentrated and extracted with CH₂Cl₂ (50 mL×3). The combined organic layers were washed with brine (20 mL×2), dried over Na₂SO₄, filtered and concentrated under reduced pressure to give a crude residue. The crude residue was then purified by column chromatography (0-100% EtOAc in hexanes) to give the title compound (752 mg, 16%). ¹H-NMR (400 MHz, CDCl₃) δ 8.48 (t, J=0.7 Hz, 1H), 8.30 (s, 1H), 7.80 (d, J=0.7 Hz, 1H), 4.07-3.94 (m, 4H), 3.03 (ddt, J=11.5, 8.3, 2.9 Hz, 1H), 2.12-1.97 (m, 2H), 1.96-1.85 (m, 2H), 1.82-1.67 (m, 4H). ES-MS [M+H]⁺=294.4.

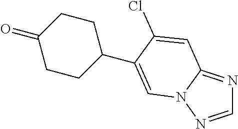

Step C. 4-(7-Chloro-[1,2,4]triazolo[1,5-a]pyridia-6-yl)eyelohexan-1-one. To a solution of 7-chloro-6-(1,4-dioxaspiro[4.5]decan-8-yl)-[1,2,4]triazolo[1,5-a]pyridine (748.6 mg, 2.55 mmol, 1.0 eq) in acetone (30 mL) and MeOH (6 mL) was added 1N HCl (20 mL, 20.0 mmol, 7.8 eq). The mixture was then stirred at room temperature for 16 h. After which time, the reaction mixture was quenched with sat. aq. NaHCO₃ (30 mL) and concentrated under reduced pressure. The reaction mixture was extracted with EtOAc (30 mL×3), washed with brine (30 mL×1), dried over Na₂SO₄, filtered and concentrated under reduced pressure to give the crude mixture of title compound (631.5 mg). This was used for the next step without further purification. ¹H-NMR (400 MHz, CDCl₃) δ 8.46 (d, J=0.8 Hz, 1H), 8.33 (s, 1H), 7.86 (d, J=0.7 Hz, 1H), 3.50 (tt, J=12.1, 3.0 Hz, 1H), 2.68-2.52 (m, 4H), 2.41 (dtd, J=11.3, 4.7, 3.1 Hz, 2H), 1.98-1.84 (m, 2H). ES-MS [M+H]⁺=250.4.

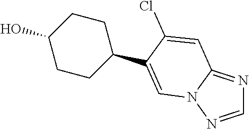

Step D. trans-4-(7-Chloro-[1,2,4]triazolo[1,5-a]pyridin-6-yl)cyclohexan-1-ol. To a solution of 4-(7-chloro-[1,2,4]triazolo[1,5-a]pyridin-6-yl)cyclohexanone (628.3 mg, 2.52 mmol, 1.0 eq) in MeOH (12. mL) was added NaBH₄ (190.4 mg, 5.03 mmol, 2.0 eq) at 0° C. Then the mixture was stirred at 0° C. for 5 h. The reaction mixture was quenched by sat. aq. NH₄Cl (5 mL), then the mixture was extracted with EtOAc (20 mL×3). The combined organic layers were washed with brine (10 mL×1), dried over Na₂SO₄, filtered and concentrated under reduced pressure to give the title compound (632.5 mg, 99%). * This sample was contaminated by trace amounts of cis-isomer by ¹H-NMR. ¹H-NMR (400 MHz, CDCl₃) δ 8.42 (d, J=0.8 Hz, 1H), 8.29 (s, 1H), 7.80 (s, 1H), 3.78-3.67 (m, 1H), 2.97 (ddt, J=11.4, 8.4, 4.5 Hz, 1H), 2.22-2.06 (m, 4H), 1.59-1.39 (m, 4H). ES-MS [M+H]⁺=252.4. * OH is not shown in ¹H-NMR spectra.

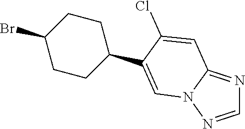

Step E. cis-6-(4-Bromocyelohexyl)-7-chloro-[1,2,4]triazolo[1,5-a]pyridine. To a solution of 4-(7-chloro-[1,2,4]triazolo[1,5-a]pyridin-6-yl)cyclohexanol (100.2 mg, 0.40 mmol, 1.0 eq) in CH₂Cl₂ (4 mL) was added CBra (185 mg, 0.56 mmol, 1.4 eq). After 5 min, Ph₃P (209 mg, 0.8 mmol, 2.0 eq) was added. The resulting mixture was stirred at room temperature for 16 h under N₂ atmosphere. After which time, the reaction mixture was concentrated, filtered, and the solid residue was washed with minimum amount of CH₂Cl₂ mL) to give the title compound (85.2 mg, 68%). ¹H-NMR (400 MHz, CDCl₃) δ 8663 (s, 2H), 8.43 (s, 1H), 4.73 (s, 1H), 3.08 (s, 1H), 2.25 (d, J=10.3 Hz, 2H), 2.01 (s, 4H), 1.89 (s, 2H). ES-MS [M+H]⁺=316.2.

### Intermediate Example 3. trans-4-(7-Methyl-[1,2,4]triazolo[1,5-a]pyridin-6-yl)cyclohexyl 4-methylbenzenesulfonate

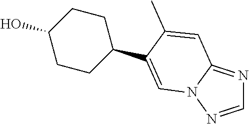

Step A. trans-4-(7-Methyl-[1,2,4]triazolo[1,5-a]pyridin-6-yl)cyclohexan-1-ol. This was prepared similar to intermediate Example 2 steps A, B, C, and D using 6-bromo-7-methyl-[1,2,4]triazo[1,5-a]pyridine instead of 6-bromo-7-chloro-[1,2,4]triazolo[1,5-a]pyridine. The crude residue was purified by reverse phase HPLC (15-95% CH₃CN in 0.1% TFA aqueous solution) to give the title compound (61.4 mg, 48%). * This sample was contaminated by 25% of cis-isomer by ¹H-NMR. ¹H-NMR (400 MHz, CDCl₃) δ 8.36 (s, 1H), 8.25 (s, 1H), 7.54 (s, 1H), 3.75 (tt, J=9.9, 4.6 Hz, 1H), 2.70 (dtd, J=11.4, 5.9, 3.0 Hz, 1H), 2.47 (s, 3H), 2.20-2.12 (m, 2H), 2.01-1.99 (m, 2H), 1.53-1.47 (m, 4H). * OH is not shown in ¹H-NMR spectra. ES-MS [M+H]⁺=232.

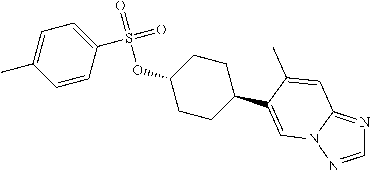

Step B. trans-4-(7-Methyl-[1,2,4]triazolo[1,5-a]pyridin-6-yl)cyclohexyl 4-methylbenzenesulfonate. To a solution of 4-(7-methyl-[1,2,4]triazolo[1,5-a]pyridin-6-yl)cyclohexanol (61.4 mg, 0.27 mmol, 1 eq) in pyridine (1 mL) at 0° C. was added tosyl chloride (60.7 mg. 0.32 mmol, 1.2 eq). The reaction mixture was stirred at 0° C. for 1 h and at room temperature overnight. The pyridine was evaporated under reduced pressure and the residue was partitioned between CH₂Cl₂ (5 mL) and H₂O (2 mL). The aqueous layer was extracted with CH₂Cl₂ (10 mL×3) and the combined organics were dried over Na₂SO₄, filtered and evaporated under reduced pressure. The crude residue was purified by column chromatography (0-100% EtOAc in hexanes) to give the title compound (69 mg, 67%). * This sample was contaminated by 6.5% of cis-isomer by ¹H-NMR. ¹H-NMR (400 MHz, CDCl₃) δ 8.30 (s, 1H), 8.24 (s, 1H), 7.83 (d, J=8.3 Hz, 2H), 7.53 (s, 1H), 7,36 (d, J=8.2 Hz, 2H), 4.50 (td, L=11.2, 5.6 Hz, 1H), 2.73-2.63 (m, 1H), 2.46 (s, 3H), 2.44 (s, 3H), 2.20-2.11 (m, 2H), 1.98 (d, J=13.5 Hz, 2H), 1.69 (qd, J=13.0, 3.3 Hz, 2H), 1.53-1.39 (m, 2H). ES-MS [M+H]⁺=386.

### c. Preparation of Representative Compounds

### Example 1. trans-2-((4-(7-Chloro-[1,2,4]triazolo[1,5-a]pyridin-6-yl)cyclohexyl)sulfonyl)-5-methyl-1,3,4-thiadiazole

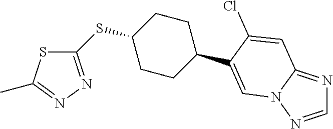

Step A. trans-2-((4-(7-Chloro-[1,2,4]triazolo[1,5-a]pyridin-6-yl)cyclohexyl)thio)-5-methyl-1,3,4-thiadiazole. To a solution of 6-(4-bromocyclohexyl)-7-chloro-[1,2,4]triazolo[1,5-a]pyridine (150 mg, 476.8 umol, 1 eq) and 5-methyl-1,3,4-thiadiazole-2-thiol (63 mg, 476.8 umol, 1 eq) in DMF (2 mL) was added K₂CO₃ (131.8 mg, 953.6 umol, 2 eq). Then the reaction mixture was stirred at 70° C. for 4 h under N₂ atmosphere. The reaction mixture was quenched with H₂O (5 mL), then the mixture was extracted with EtOAc (5 mL×3) The combined organic layers were washed with brine (10 mL), dried over Na₂SO₄, filtered and concentrated under reduced pressure to give a crude residue. The residue was purified by preparative-TLC to give the title compound (90 mg). ¹H-NMR (400 MHz, CDCl₃) δ 8.45 (s, 1H), 8.03 (s, 1H), 7.94 (s, 1H), 3.87 (tt, J=12.1, 3.8 Hz, 1H), 3.04-3.11 (m, 1H), 2.71-2.78 (m, 3H), 2.44-2.52 (m, 2H), 2.14-2.23 (m, 2H), 1.54-1.82 (m, 4H). ES-MS [M+H]⁺=366.

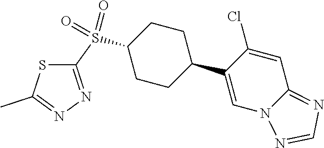 [SMILES: Cc1nnc(S(=O)(=O)[C@H]2CC[C@H](c3cn4ncnc4cc3Cl)CC2)s1]

Step B. trans-2-((4-(7-Chloro[1,2,4]triazolo[1,5-a]pyridin-6- yl)cyclohexyl)sulfonyl)-5-methyl-1,3,4-thiadiazole. To a solution of trans-2-((4-(7-chloro-[1,2,4]triazolo[1,5-a]pyridin-6-yl)cyclohexyl)thio)-5-methyl-1,3,4-thiadiazole (90 mg, 246 nmol, 1 eq) in CH₂Cl₂ (2 mL) was added a solution of m-CPBA (250 mg, 1.23 mmol, 85% purity, 5 eq) in CH₂Cl₂ (2 mL) at 0° C. The mixture was then stirred at 0° C. for 2 h, after which time the mixture was stirred at room temperature for 2 h. The reaction mixture was quenched with H₂O (5 mL), then the mixture was extracted with EtOAc (5 mL×3). The combined organic layers were washed with brine (10 mL), dried over Na₂SO₄, filtered and concentrated under reduced pressure to give a crude residue, which was purified by preparative-HPLC (column: Waters Xbridge BEH C18 100*30 mm*10 μm; mobile phase: [water(10 mM NH₄HCO₃)—CH₃CN]; B %: 12-42%, 8 min.) to give the title compound (40 mg, 40%). ¹H-NMR (400 MHz, CDCl₃) δ 8.41 (s, 1H), 8.32 (s, 1H), 7.83 (s, 1H), 3.66 (tt, J=12.5, 3.6 Hz, 1H), 3.05 (tt, J=12.1, 2.9 Hz, 1H), 2.94 (s, 3H), 2.41-2.49 (m, 2H), 2.26-2.34 (m, 2H), 1.88-2.02 (m, 2H), 1.45-1.56 (m, 2H). ES-MS [M+H]⁺=398.

### Example 2. trans-2-((4-(7-chloro[1,2,4]triazolo[1,5-a]pyridin-6-yl)cyclohexyl)sulfonyl)-5-methyl-1,3,4-oxadiazole

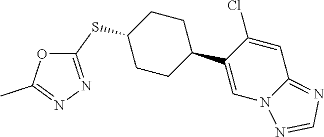 [SMILES: Cc1nnc(S[C@H]2CC[C@H](c3cn4ncnc4cc3Cl)CC2)o1]

Step A. trans-2-((4-(7-Chloro[1,2,4]triazolo[1,5-a]pyridin-6-yl)cyclohexyl)thio)-5-methyl-1,3,4-oxadiazole. To a solution of 6-(4-bromocyclohexyl)-7-chloro-[1,2,4]triazolo[1,5-a]pyridine (13.1 mg, 0.04 mmol, 1 eq) and 5-methyl-1,3,4-oxadiazole-2-thiol (14.5 mg, 0.12 mmol, 3 eq) in DMF (0.5 mL) was added K₂CO₃ (12 mg, 0.9 mmol, 3 eq) at room temperature. The reaction mixture was then heated to 70° C. for 1 h, after which time the reaction mixture was quenched by H₂O (0.5 mL) and extracted with EtOAc (3 mL×3). The organic layer was washed with brine (2 mL×1). The organic layer was passed through a phase separator and concentrated under reduced pressure to give the crude residue. The crude residue was then purified by RP-HPLC (5-95% CH₃CN in 0.1% NH₄OH aqueous solution) to give the title compound (5.1 mg, 35%). ¹H-NMR (400 MHz, CDCl₃) δ 8.42 (s, 1H), 8.32 (s, 1H), 7.83 (s, 1H), 3.71 (tt, J=12.3, 3.9 Hz, 1H), 3.06 (tt, J=11.9, 3.1 Hz, 1H), 2.53 (s, 3H), 2.51-2.43 (m, 2H), 2.25-2.14 (m, 2H), 1.77 (qd, J=12.8, 3.2 Hz, 2H), 1.60 (qd, J=12.9, 3.1 Hz, 2H). ES-MS [M+H]⁺=350.2.

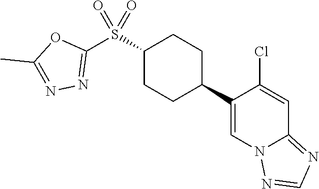 [SMILES: Cc1nnc(S(=O)(=O)[C@H]2CC[C@H](c3cn4ncnc4cc3Cl)CC2)o1]

Step B. trans-2-((4-(7-chloro-[1,2,4]triazolo[1,5-a]pyridin-6-yl)cyclohexyl)sulfonyl)-5-methyl-1,3,4-oxadiazole. To a solution of trans-2-((4-(7-chloro-[1,2,4]triazolo[1,5-a]pyridin-6-yl)cyclohexyl)thio)-5-methyl-1,3,4-oxadiazole (5.1 mg, 14 μmol, 1 eq) in CH₂Cl₂ (1.0 mL) was added m-CPBA (12.3 mg, 70 μmol, 5 eq) at room temperature. After 16 h, the reaction mixture was quenched with H₂O (2 mL) and extracted with CH₂Cl₂ (5 mL×2). The combined extracts were passed through a phase separator and concentrated under reduced pressure. The crude residue was purified by reverse phase HPLC (15-95% CH₃CN in 0.1% TFA aqueous solution) to give the title compound (1.6 mg, 29%). ¹H-NMR (400 MHz, CDCl₃) δ 8.41 (d, J=0.9 Hz, 1H), 8.32 (s, 1H), 7.84 (d, J=0.6 Hz, 1H), 3.56 (tt, J=12.5, 3.6 Hz, 1H), 3.06 (tt, J=12.1, 3.1 Hz, 1H), 2.71 (s, 3H), 2.52-2.44 (m, 2H), 2.33 (d, J=13.3 Hz, 2H), 1.95 (qd, J=13.0, 3.6 Hz, 2H), 1.52 (td, J=12.8, 3.3 Hz, 2H). ES-MS [M+H]⁺=382.4.

### Example 3. cis-2-Methyl-5-((4-(7-methyl-[1,2,4]triazolo[1,5-a]pyridin-6-yl)cyclohexyl)sulfonyl)-1,3,4-oxadiazole

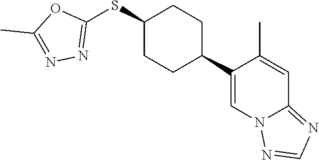 [SMILES: Cc1nnc(S[C@H]2CC[C@@H](c3cn4ncnc4cc3C)CC2)o1]

Step A. cis-2-Methyl-5-((4-(7-methyl-[1,2,4]triazolo[1,5-a]pyridin-6-yl)cyclohexyl)thio)-1,3,4-oxadiazole. To a solution of 5-methyl-1,3,4-oxadiazole-2-thiol (27.1 mg, 0.23 mmol, 3 eq) and trans-4-(7-methyl-[1,2,4]triazolo[1,5-a]pyridin-6-yl)cyclohexyl 4-methylbenzenesulfonate (30 mg, 0.08 mmol, 1 eq) in DMF (1 mL) was added Cs₂CO₃ (51 mg, 0.16 mmol, 2 eq). The reaction mixture was stirred at 70° C. overnight, after which time the reaction mixture was filtered and purified by reverse phase HPLC (12-95% CH₃CN in 0.1% TFA aqueous solution) to give the title compound (13.8 mg, 53%). ¹H-NMR (400 MHz, CDCl₃) δ 8.43 (s, 1H), 8.24 (s, 1H), 7.51 (s, 1H), 4.39 (p, J=3.4 Hz, 1H), 2.78 (tt, J=11.6, 3.5 Hz, 1H), 2.52 (s, 3H), 2.47-2.44 (m, 3H), 2.30 (d, J=14.7 Hz, 2H), 2.07 (tt, J=14.7, 3.8 Hz, 2H), 1.91-1.74 (m, 4H). ES-MS [M+H]⁺=330.

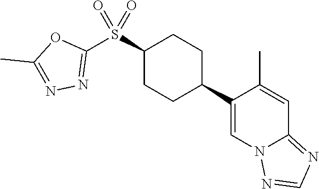 [SMILES: Cc1nnc(S(=O)(=O)[C@H]2CC[C@@H](c3cn4ncnc4cc3C)CC2)o1]

Step B. cis-2-Methyl-5-((4-(7-methyl[1,2,4]triazolo[1,5-a]pyridin-6-yl)cyclohexyl)sulfonyl)-1,3,4-oxadiazole. To a solution of cis-2-methyl-5-((4-(7-methyl-[1,2,4]triazolo[1,5-a]pyridin-6-yl)cyclohexyl)thio)-1,3,4-oxadiazole (13.8 mg, 0.04 mmol, 1 eq) in CH₂Cl₂ (1 mL) was added in-CPBA (36 mg, 0.2.1 mmol, 5 eq) at 0° C. Then, the reaction mixture was warmed to room temperature and stirred for 2 h. The reaction mixture was then quenched with H₂O (2 mL) and extracted with CH₂Cl₂ (5 mL×2). The combined extracts were passed through a phase separator and concentrated under reduced pressure. The crude residue was purified by reverse phase HPLC (15-95% CH₃CN in 0.1% TFA aqueous solution) to give the title compound (8.1 mg, 53%). ¹H-NMR (400 MHz, CDCl₃) δ 8.46 (s, 1H), 8.27 (s, 1H), 7.55 (s, 1H), 3.86 (dt, J=4.9, 2.8 Hz, 1H), 2.89-2.78 (m, 1H), 2.70 (s, 3H), 2.69-2.67 (m, 2H), 2.47 (s, 3H), 2.19-1.99 (m, 4H), 1.92 (m, 2H). ES-MS [M+H]⁺=362.

The compounds shown in Table 2 may be prepared similarly to the compound described above, with appropriate starting materials.

TABLE 2

| No. | STRUCTURE | NAME | ¹H-NMR and/or ES-MS [M + H]⁺ |
|---|---|---|---|
| 1 | 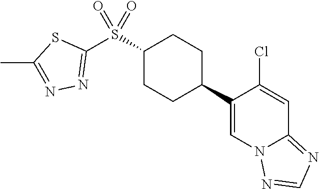 [SMILES: Cc1nnc(S(=O)(=O)[C@H]2CC[C@H](c3cn4ncnc4cc3Cl)CC2)s1] | trans-2-((4-(7-chloro- [1,2,4]triazolo[1,5- a]pyridin-6- yl)cyclohexyl)sulfonyl)- 5-methyl-1,3,4- thiadiazole | ¹H-NMR (400 MHz, CDCl₃) δ 8.41 (s, 1H), 8.32 (s, 1H), 7.83 (s, 1H), 3.66 (tt, J = 12.5, 3.6 Hz, 1H), 3.05 (tt, J = 12.1, 2.9 Hz, 1H), 2.94 (s, 3H), 2.41-2.49 (m, 2H), 2.26- 2.34 (m, 2H), 1.88-2.02 (m, 2H), 1.45- 1.56 (m, 2H). ES-MS [M + H]⁺ = 398 |
| 2 | 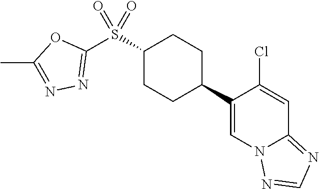 [SMILES: Cc1nnc(S(=O)(=O)[C@H]2CC[C@H](c3cn4ncnc4cc3Cl)CC2)o1] | trans-2-((4-(7-chloro- [1,2,4]triazolo[1,5- a]pyridin-6- yl)cyclohexyl)sulfonyl)- 5-methyl-1,3,4- oxadiazole | ¹H-NMR (400 MHz, CDCl₃) δ 8.41 (d, J = 0.9 Hz, 1H), 8.32 (s, 1H), 7.84 (d, J = 0.6 Hz, 1H), 3.56 (tt, J = 12.5, 3.6 Hz, 1H), 3.06 (tt, J = 12.1, 3.1 Hz, 1H), 2.71 (s, 3H), 2.52- 2.44 (m, 2H), 2.33 (d, J = 13.3 Hz, 2H), 1.95 (qd, J = 13.0, 3.6 Hz, 2H), 1.52 (td, J = 12.8, 3.3 Hz, 2H). ES-MS [M + H]⁺ = 382.4. |
| 3 | 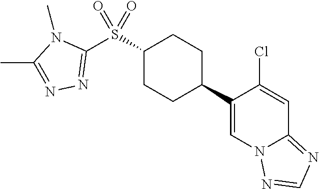 [SMILES: Cc1nnc(S(=O)(=O)[C@H]2CC[C@H](c3cn4ncnc4cc3Cl)CC2)n1C] | trans-7-chloro-6-(4- ((4,5-dimethyl-4H- 1,2,4-triazol-3- yl)sulfonyl)cyclohexyl)- [1,2,4]triazolo[1,5- a]pyridine | ¹H-NMR (400 MHz, CDCl₃) δ 8.43 (s, 1H), 8.34 (s, 1H), 7.87 (s, 1H), 3.88 (s, 3H), 3.82 (ddd, J = 12.5, 8.9, 3.7 Hz, 1H), 3.07 (tt, J = 11.8, 2.8 Hz, 1H), 2.54 (s, 3H), 2.49 (s, 2H), 2.30 (d, J = 12.0 Hz, 2H), 1.95 (qd, J = 13.0, 3.4 Hz, 2H), 1.57 (qd, J = 13.1, 3.0 Hz, 2H). ES-MS [M + H]⁺ = 395. |
| 4 | 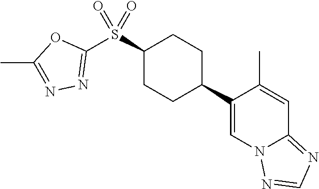 [SMILES: Cc1nnc(S(=O)(=O)[C@H]2CC[C@@H](c3cn4ncnc4cc3C)CC2)o1] | cis-2-methyl-5-((4-(7- methyl- [1,2,4]triazolo[1,5- a]pyridin-6- yl)cyclohexyl)sulfonyl)- 1,3,4-oxadiazole | ¹H-NMR (400 MHz, CDCl₃) δ 8.46 (s, 1H), 8.27 (s, 1H), 7.55 (s, 1H), 3.86 (dt, J = 4.9, 2.8 Hz, 1H), 2.89-2.78 (m, 1H), 2.70 (s, 3H), 2.69-2.67 (m, 2H), 2.47 (s, 3H), 2.19- 1.99 (m, 4H), 1.92 (m, 2H). ES-MS [M + H]⁺ = 362. |

### Biological Activity

### Cell-Based Functional Assay of Muscarinic Acetylcholine Receptor Activity

All functional cell-based assays were performed essentially as previously described (Mario et al., Mol. Pharm. 2009, 75(3), 577-588; Brady et al., J. Pharm. & Exp. Ther. 2008, 327, 941-953). Initial, single point (10 μM) compound characterization was performed in stable Chinese Hamster Ovary (CHO) cell lines constitutively expressing human M₅ receptors. These were plated at 15,000 cells per 20 μL per well in Greiner 384-well black-walled, TC-treated, clear-bottomed plates (Fisher) in Ham's F12 medium supplemented with 10% FBS and 20 mM HEPES. Cells were incubated overnight at 37° C. under 5% CO₂. The following day, medium was exchanged with assay buffer (Hank's Balanced Salt Solution supplemented with 20 mM HEPES and 2.5 mM Probenecid, pH 7.4) leaving 20 μL of assay buffer in each well. This was followed by the addition of 20 μL of 2.3 μM of Fluo-4 AM (Invitrogen) in assay buffer (final concentration 1.15 μM). The cells were then incubated 50 minutes at 37° C. under 5% CO₂. The assay buffer plus dye was then exchanged with fresh assay buffer leaving a volume of 20 μL in each well. Test compounds were diluted into assay buffer to a 2× (20 μM) concentration in 0.2% dimethylsulfoxide (DMSO) in columns 3-22 with matching DMSO concentration in columns 1, 2, 23, and 25; compounds were added to the assay for a final concentration of 10 μM and a final DMSO concentration of 0.1%. Acetylcholine (Sigma-Aldrich) was prepared to provide 5× concentrations of EC₂₀, EC₈₀, and ECₘₐₓ in the triple-addition assay, providing a signal window to view agonism, potentiation, and inhibition of the acetylcholine response as well as a means to normalize to the maximum acetylcholine response.

Either an FDSS (Hamamatsu) or Panoptic (WaveFront Biosciences) kinetic imaging plate reader was used for assay execution and measurement of calcium flux. After establishing baseline fluorescence, test compounds (20 μL) were added to the cells using the reader's integrated pipettor and allowed to equilibrate for 140 seconds before addition of the EC₂₀ concentration of acetylcholine (10 μL) along with vehicle in selected DMSO-only wells in the outer two columns. The EC₈₀ concentration of acetylcholine (10 μL) was added 125 seconds after the EC₂₀ addition along with ECₘₐₓ concentration in the wells receiving vehicle in the second addition. The raw fluorescence data from each well was normalized to the corresponding initial fluorescence reading (static ratio). The maximum fluorescence value following each addition was determined and the minimum value within that same timeframe was subtracted for each well then normalized to the average of the ECₘₐₓ maximum-minimum response, providing a % AChₘₐₓ value for each addition for each well. The single point values represent mean values determined within the EC₈₀ addition timeframe obtained from at least three independent determinations performed in triplicate or greater (error bars represent +/−SEM) unless otherwise specified.

Further characterization of test compounds (compound potency and mAChR subtype-selectivity) was performed on the FDSS with calcium mobilization assays performed as previously described (Mario et al., 2009; Brady et al., 2008) and in a format similar to that described above using the same reagents. CHO cells stably expressing hM₁, hM₂/G_{qi5}, hM₃, hM₄/G_{qi5}, hM₅, rM₁, rM₂/G_{qi5}, rM₃, rM₄/G_{qi5}, or rM₅ were plated in the manner described above. A ten-point concentration range of test compound was serial diluted in assay buffer to 2× final concentration and acetylcholine was diluted in assay buffer to 5× of the EC₂₀ and EC₈₀ concentrations, determined empirically and 5× maximal (2 mM final concentration) stock concentrations. FDSS protocols were carried out as described above; the static ratio was calculated and the minimum response subtracted from the maximum response within the timeframe of each addition. This max-min response was then normalized to the maximum acetylcholine response. Calculation of IC₅₀ was performed using the percent maximum acetylcholine response for the EC₈₀ addition through the Vortex and Studies modules of the Dotmatics data management software. Results are stored in the Dotmatics database and an audit trail of any changes to their analysis is generated. Data shown represent mean values obtained from at least three independent determinations performed in triplicate or greater (error bars represent ±SEM) unless otherwise specified.

TABLE 3
Activity of Compounds in a mAChR M₅ Cell-Based Assay

| No. | IC₅₀ (μM) | EC₈₀ MIN (%) | CELL LINE |
|---|---|---|---|
| 1 | 0.185 | 3 | hM₅ |
| 2 | 0.532 | 3 | hM₅ |
| 3 | 0.033 | 2 | hM₅ |
| 4 | >10 | 55 | hM₅ |

We claim:

1. A compound of formula (I), or a pharmaceutically acceptable salt thereof, 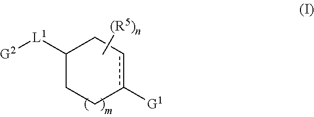
wherein:
“ [symbol] ” is a single or double bond;
m is 0 or 1;
L¹ is SO₂ or SO;
G¹ is a 9- to 12-membered heteroaryl, G¹ being attached at an aromatic ring carbon atom, wherein G¹ is optionally substituted with 1-5 substituents independently selected from the group consisting of oxo, halogen, C₁₋₆alkyl, C₁₋₆haloalkyl, C₂₋₆alkenyl, —OR^{1a}, —NR^{1a}R^{1b}, —SR^{1a}, —NR^{1a}C(O)R^{1c}, cyano, —C(O)OR^{1a}, —C(O)NR^{1a}R^{1b}, —C(O)NR^{1c}, —SO₂R^{1d}, —SO₂NR^{1a}R^{1b}, G^{1a}, —C₁₋₃alkylene-G^{1a}, and —C₁₋₃alkylene-Y¹;
G² is a 6- to 12-membered aryl or 5- to 12 membered heteroaryl, each optionally substituted with 1-5 substituents independently selected from the group consisting of halogen, C₁₋₆alkyl, C₁₋₆haloalkyl, oxo, —OR^{2a}, —NR^{2a}R^{2b}, —SR^{2a}, —NR^{2a}C(O)R^{2c}, cyano, —C(O)OR^{2a}, —C(O)NR^{2a}R^{2b}, —C(O)R^{2c}, —SO₂R^{2d}, —SO₂NR^{2a}R^{2b}, G^{2a}, —C₁₋₃alkylene-G^{2a}, and —C₁₋₃alkylene-Y²;
R^{1a}, R^{1b}, and R^{1c}, at each occurrence, are each independently hydrogen, C₁₋₆alkyl, C₁₋₆haloalkyl, G^{1a}, or —C₁₋₃alkylene-G^{1a};
R^{1d}, at each occurrence, is independently C₁₋₆alkyl, C₁₋₆haloalkyl, G^{1a}, or —C₁₋₃alkylene-G^{1a};
R^{2a}, R^{2b}, and R^{2c}, at each occurrence, are each independently hydrogen, C₁₋₆alkyl, C₁₋₆haloalkyl, G^{2a}, or —C₁₋₃alkylene-G²;
R^{2d}, at each occurrence, is independently C₁₋₆alkyl, C₁₋₆haloalkyl, G^{2a}, or —C₁₋₃alkylene-G^{2a};
G^{1a} and G^{2a}, at each occurrence, are independently a C₃₋₈cycloalkyl, a 4- to 12-membered heterocyclyl, a 6- to 12-membered aryl, or a 5- to 12-membered heteroaryl, wherein G^{1a} and G^{2a} are independently optionally substituted with 1-5 substituents independently selected from the group consisting of halogen, oxo, C₁₋₄alkyl, —OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Y¹ and Y², at each occurrence, are independently —OC₁₋₄alkyl, —OC₁₋₄haloalkyl, OH, NH₂, —NHC₁₋₄alkyl), —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
R⁵, at each occurrence, is independently halogen, cyano, oxo, C₁₋₄alkyl, C₁₋₄haloalkyl, —OR^{5a}, or C₃₋₄cycloalkyl,
R^{5a}, at each occurrence, is independently hydrogen, C₁₋₄alkyl, C₁₋₄haloalkyl, C₃₋₄cycloalkyl, or —C₁₋₃alkylene-C₃₋₄cycloalkyl; and
n is 0, 1, 2, 3, 4, or 5;
provided the compound is not 2,8,9-trimethyl-5-[4-(phenylsulfonyl)cyclohexyl]thieno[3,2-e][1,2,4]triazolo[1,5-c]pyrimidine.

2. The compound of claim 1, or a pharmaceutically acceptable salt thereof, wherein the ring system of the optionally substituted 9- to 12-membered heteroaryl at G¹ is a 9-membered fused bicyclic heteroaryl having four double bonds and two to four nitrogen ring atoms, wherein one nitrogen atom occupies a position at the ring junction of the bicyclic ring system, G¹ being attached at a first carbon atom of G¹, wherein the first carbon atom of G¹ is in a 6-membered ring of the 9-membered fused bicyclic ring system.

3. The compound of claim 2, or a pharmaceutically acceptable salt thereof, wherein at G¹ the first carbon atom and the ring junction nitrogen atom are separated by one ring atom.

4. The compound of claim 3, or a pharmaceutically acceptable salt thereof, wherein the ring system of the optionally substituted 9- to 12-membered heteroaryl at G¹ has the following ring system: 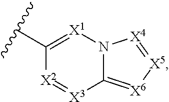
wherein x¹-x⁶ independently represent carbon or nitrogen ring atoms, provided that 1-3 of x¹-x⁶ are nitrogen atoms.

5. The compound of claim 4, or a pharmaceutically acceptable salt thereof, wherein 2 of x¹-x⁶ are nitrogen atoms.

6. The compound of claim 5, or a pharmaceutically acceptable salt thereof, wherein the ring system 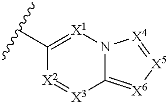
is the ring system

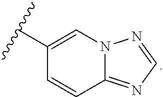 [SMILES: *c1ccc2ncnn2c1]

7. The compound of any of claims 1-4, or a pharmaceutically acceptable salt thereof, wherein G¹ is 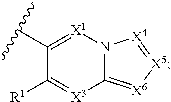
x¹, x³, x⁴, x⁵, and x⁶ are N or CH, wherein 1-3 of x¹, x³, x⁴, x⁵, and x⁶ are N;
R¹ is C₁₋₄alkyl, C₁₋₄haloalkyl, halogen, C₂₋₄alkenyl, —OC₁₋₄alkyl, —OC₁₋₄fluoroalkyl, —C(O)OR^{1a}, —C(O)NR^{1a}R^{1b}, —C₁₋₃alkylene-OH, or G^{1a};
R^{1a} and R^{1b} are each independently hydrogen or C₁₋₄alkyl; and
G^{1a} is a C₃₋₄cycloalkyl.

8. The compound of claim 7, or a pharmaceutically acceptable salt thereof, wherein R¹ is C₁₋₄alkyl or halogen.

9. The compound of claim 8, or a pharmaceutically acceptable salt thereof, wherein R¹ is methyl, fluoro, or chloro.

10. The compound of any of claims 7-9, or a pharmaceutically acceptable salt thereof, wherein G¹ is

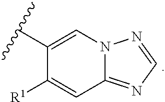

11. The compound of any of claims 1-10, or a pharmaceutically acceptable salt thereof, wherein G¹ is

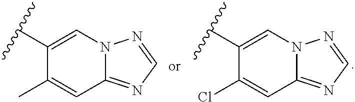 [SMILES: *c1cn2ncnc2cc1C ; *c1cn2ncnc2cc1Cl]

12. The compound of any of claims 1-11, or a pharmaceutically acceptable salt thereof, wherein G² is the optionally substituted 5- to 12 membered heteroaryl.

13. The compound of claim 12, or a pharmaceutically acceptable salt thereof, wherein the ring system of the optionally substituted 5- to 12-membered heteroaryl of G² is a 5- to 6-membered monocyclic heteroaryl ring system.

14. The compound of claim 13, or a pharmaceutically acceptable salt thereof, wherein the ring system of the optionally substituted 5- to 12-membered heteroaryl of G² is 1,2,4-triazolyl, 1,3,4-thiadiazolyl, or 1,3,4-oxadiazolyl.

15. The compound of claim 14, or a pharmaceutically acceptable salt thereof, wherein G² is

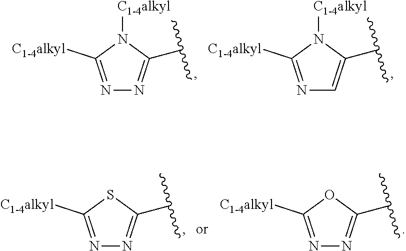

16. The compound of any of claims 1-15, or a pharmaceutically acceptable salt thereof, wherein L¹ is SO₂.

17. The compound of any of claims 1-16, or a pharmaceutically acceptable salt thereof, wherein n is 0.

18. The compound of any of claims 1-17, wherein “ [symbol] ” is a single bond.

19. The compound of any of claims 1-18, or a pharmaceutically acceptable salt thereof, wherein m is 1.

20. The compound of any of claims 1-19, or a pharmaceutically acceptable salt thereof, wherein the compound of formula (I) has formula (I-A):

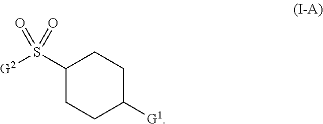

21. The compound of claim 20, or a pharmaceutically acceptable salt thereof, wherein formula (I-A) has trans stereochemistry, i.e., formula (I-A1)

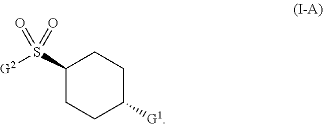

22. The compound of claim 1, selected from the group consisting of:
trans-2-((4-(7-chloro-[1,2,4]triazolo[1,5-a]pyridin-6-yl)cyclohexyl)sulfonyl)-5-methyl-1,3,4-thiadiazole;
trans-2-((4-(7-chloro-[1,2,4]triazolo[1,5-a]pyridin-6-yl)cyclohexyl)sulfonyl)-5-methyl-1,3,4-oxadiazole;
trans-7-chloro-6-(4-((4,5-dimethyl-4H-1,2,4-triazol-3-yl)sulfonyl)cyclohexyl)-[1,2,4]triazolo[1,5-a]pyridine; and
cis-2-methyl-5-((4-(7-methyl[1,2,4]triazolo[1,5-a]pyridin-6-yl)cyclohexyl)sulfonyl)-1,3,4-oxadiazole;
or a pharmaceutically acceptable salt thereof.

23. A pharmaceutical composition comprising the compound of any of claims 1-22, or a pharmaceutically acceptable salt thereof, and a pharmaceutically acceptable carrier.

24. A method of treating a psychiatric disorder comprising administering to a subject in need thereof, a therapeutically effective amount of the compound of any of claims 1-22, or a pharmaceutically acceptable salt thereof, or the pharmaceutical composition of claim 23.

25. The method of claim 24, wherein the psychiatric disorder is selected from the group consisting of substance-related disorders, opioid-related disorders, alcohol-related disorders, sedative-, hypnotic-, or anxiolytic-related disorders, stimulant-related disorders, cannabis-related disorders, hallucinogen-related disorders, inhalant-related disorders, tobacco-related disorders, depressive disorders, persistent depressive disorder (dysthymia), anxiety disorders, schizophrenia, psychotic disorder NOS, brief psychotic disorder, schizophreniform disorder, schizoaffective disorder, delusional disorder, shared psychotic disorder, catastrophic schizophrenia, postpartum psychosis, psychotic depression, psychotic break, tardive psychosis, myxedematous psychosis, occupational psychosis, menstrual psychosis, secondary psychotic disorder, bipolar I disorder with psychotic features, and substance-induced psychotic disorder.

26. A method of inhibiting mAChR M₅ comprising administering to a subject in need thereof, a therapeutically effective amount of the compound of any of claims 1-22, or a pharmaceutically acceptable salt thereof, or the pharmaceutical composition of claim 23.
